(19) United States
(12) Patent Application Publication
(10) Pub. No.: US 2025/0282761 A1
(43) Pub. Date: Sep. 11, 2025

(54) SALT AND CRYSTAL FORMS OF AN EPIDERMAL GROWTH FACTOR RECEPTOR INHIBITOR

(71) Applicant: Blueprint Medicines Corporation, Cambridge, MA (US)

(72) Inventors: Eric Gruff, Cambridge, MA (US); Shanming Kuang, South Plainfield, NJ (US); Anjali Pandey, Cambridge, MA (US); Harsh Shah, South Plainfield, NJ (US); Tian Xie, South Plainfield, NJ (US)

(21) Appl. No.: 18/862,859

(22) PCT Filed: May 4, 2023
(86) PCT No.: PCT/US2023/020925

§ 371 Date: Nov. 4, 2024

Related U.S. Application Data
(60) Provisional application No. 63/338,371, filed on May 4, 2022.

(51) Int. Cl. C07D 403/12; A61K 31/506

(52) U.S. Cl. CPC C07D 403/12; A61K 31/506

(57) ABSTRACT

Various salt forms and free base solid forms of Compound (I) represented by the following formula are disclosed. Pharmaceutical compositions comprising the same, methods of treating a disease associated with an epidermal growth factor receptor (EGFR) family kinase using the same, and methods for making the salt forms of Compound (I) and crystalline forms thereof are also disclosed.

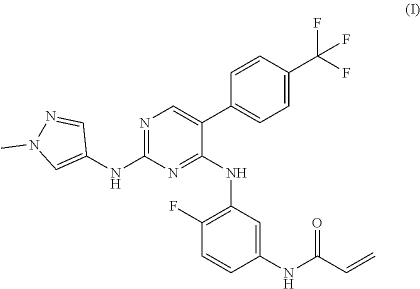 [SMILES: C=CC(=O)Nc1ccc(F)c(Nc2nc(Nc3cnn(C)c3)ncc2-c2ccc(C(F)(F)F)cc2)c1]

### RELATED APPLICATIONS

The present application claims the benefit of U.S. Provisional Application Ser. No. 63/338,371 filed on May 4, 2022, the contents of which are incorporated by reference herein.

### BACKGROUND

Lung cancer is the second most common cancer worldwide and the leading cause of cancer deaths. In 2020, lung cancer accounted for over 2 million new global cancer diagnoses and over 1 million deaths. Non-small cell lung cancers (NSCLCs) make up approximately 80% of all lung cancers, with adenocarcinoma being the most common histology. While the most common causes of lung cancer are smoking and exposure to other environmental toxins, oncogenic driver mutations are frequently present and provide an opportunity for targeted therapy.

Epidermal growth factor receptor (EGFR) is a transmembrane receptor tyrosine kinase which is activated by epidermal growth factor ligand. In a subset of NSCLC and certain other tumors, specific mutations in the EGFR gene result in ligand-independent receptor activation and drive uncontrolled tumor cell survival and proliferation. The incidence of EGFR mutations in NSCLC varies by location and ethnicity. According to a study in 2021, EGFR mutations are most common in adenocarcinomas, which comprise approximately 40% of all lung cancers, and are enriched in women, Asian populations, and non-smokers. Epidermal growth factor receptor-associated lung cancers have a predilection for CNS metastases, with approximately 25% of patients having brain metastases at initial presentation and up to 50% at some time during the course of their disease.

Comprehensive genomic profiling of tumor samples from 14,483 NSCLC cases in the course of clinical care identified 2,251 cases with EGFR mutations. EGFR Exon 19 deletions (47%) and EGFR L858R (32%) were the most common EGFR mutations. Uncommon EGFR mutations were identified, such as G719X (4%), L861Q (2%), and S768I (1%), as well as cases with compound EGFR-activating mutations (2%). Two hundred and sixty-three of the 2,251 mutant EGFR cases harbored Exon 20 insertion, representing 12% of all EGFR-mutant NSCLC and 1.8% of all NSCLC, making it the third most common type of oncogenic EGFR mutation (Riess J W, et al. J Thorac Oncol. 2018; 13:1560-8).

Epidermal growth factor receptor Exon 20 insertion mutations (Ex20ins) are characterized by inframe mutations leading to insertions of 1 to 7 amino acids across a span of approximately 15 amino acids. In addition to NSCLC, EGFR Ex20ins are found in a small percentage of urothelial and endometrial cancers, glioblastomas, sinonasal cancers, and pediatric bithalamic gliomas. Like other EGFR-mutated NSCLCs, approximately one quarter of patients with EGFR Ex20ins have brain metastases at the time of initial presentation.

Because of their unique structural characteristics, EGFR Ex20ins are generally insensitive to the first three generations of EGFR tyrosine kinase inhibitors (TKIs). Standard of care for first-line metastatic disease therefore remains platinum-based chemotherapy. The role of immune checkpoint inhibitors remains poorly defined. For patients with EGFR Ex20ins and progression after platinum-based chemotherapy, standard of care has recently changed with the approval of two agents, amivantamab and mobocertinib. Both agents received accelerated approval from the US FDA as treatment for NSCLC with EGFR Ex20ins that has progressed on or after platinum-based chemotherapy. Although the development of targeted TKIs such as amivantamab and mobocertinib for NSCLC with EGFR mutations has led to significant improvement in patient outcomes, there still remains a significant unmet need, particularly for patients with EGFR Ex20ins. Over half of patients treated with either amivantamab or mobocertinib in clinical trials did not achieve an objective response, and both agents have yet to demonstrate a survival benefit. Further, neither drug has demonstrated meaningful CNS activity, and tolerability remains a concern. Thus, there is a need for an EGFR Ex20ins-targeted TKI that is highly CNS penetrant, demonstrates potent activity against a range of EGFR Ex20ins, and is EGFR WT sparing leading to an improved side effect profile to help address this unmet need.

International Patent Application No. PCT/US2021/057472, the entire teachings of which are incorporated herein by reference, discloses selective inhibitors of EGFR, including exon 20 mutant proteins, which can be used to treat various cancers. The structure of one of the inhibitors disclosed in PCT Patent Application No. PCT/US2021/057472, referred to herein as “Compound (I)” is shown below:

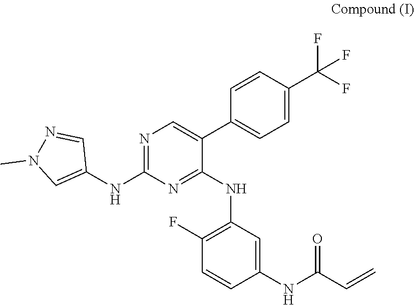 [SMILES: C=CC(=O)Nc1ccc(F)c(Nc2nc(Nc3cnn(C)c3)ncc2-c2ccc(C(F)(F)F)cc2)c1]

There is a need to develop new salt forms and/or solid forms of Compound (I) that are suitable to large scale manufacture and commercialization.

### SUMMARY

The present disclosure is directed to i) novel pharmaceutically acceptable mesylate salts of Compound (I) having different solid forms; and ii) novel crystalline free bases of Compound (I) having different solid forms.

It has now also been found that 1:1 mesylate salt of Compound (I) can be crystallized under well-defined conditions to provide desired crystalline forms which have good thermal behavior with high melting point onsets and are suitable for large scale synthesis. Minimal mass loss was observed during thermogravimetric analysis.

Moreover, four different crystalline forms (Form A, Form B, Form H, and Form I) of the mesylate salt of Compound (I) have been identified. Among these crystalline forms, Form A, Form B and Form H are anhydrates, and Form I is a hydrate.

Mesylate Form B, Form H and Form I showed promising solid state characterization results. They are physically and chemically (>99.8% assay purity) stable at 25° C./60% relative humidity (RH), 40°/75% RH and 60° C. up to 2 weeks, which suggested that all three forms are physicochemically stable. See Examples 2-5.

In one aspect, the present disclosure provides a mesylate salt of Compound (I) represented by the structural formula:

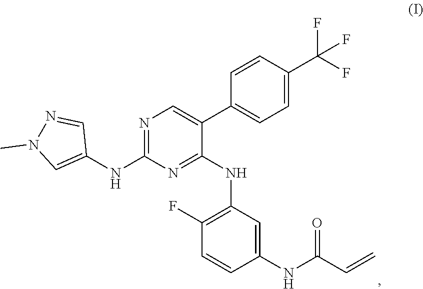 [SMILES: C=CC(=O)Nc1ccc(F)c(Nc2nc(Nc3cnn(C)c3)ncc2-c2ccc(C(F)(F)F)cc2)c1]

wherein the molar ratio between Compound (I) and methanesulfonic acid is 1:1. In some embodiments the mesylate salt is a crystalline salt.

In another aspect, the present disclosure provides a crystalline mesylate salt of Compound (I) wherein the crystalline mesylate salt is a crystalline Form A.

In another aspect, the present disclosure provides a crystalline mesylate salt of Compound (I) wherein the crystalline mesylate salt is a crystalline Form B.

In another aspect, the present disclosure provides a crystalline mesylate salt of Compound (I) wherein the crystalline mesylate salt is a crystalline Form H.

In another aspect, the present disclosure provides a crystalline mesylate salt of Compound (I) wherein the crystalline mesylate salt is a crystalline Form I.

In another aspect, the present disclosure provides a first crystalline polymorph of the free base of Compound (I). This first polymorph is referred to herein as the “Crystalline Form A.”

In another aspect, the present disclosure provides a second crystalline polymorph of the free base of Compound (I). This second polymorph is referred to herein as the “Crystalline Form B.”

In another aspect, the present disclosure provides a pharmaceutical composition comprising a mesylate salt of Compound (I), or one of the mesylate crystalline forms disclosed herein, or comprising a crystalline form of Compound (I) free base as disclosed herein, and a pharmaceutically acceptable carrier.

The present disclosure also provides a method of treating a disease associated with an EGFR in a subject in need thereof, comprising administering to the subject a therapeutically effective amount of a mesylate salt of Compound (I) or one of the mesylate crystalline forms disclosed herein, or administering a crystalline polymorph of Compound (I) free base as disclosed herein. In some embodiments, the disease in the subject is characterized by an EGFR mutation.

The present disclosure also provides a method of treating cancer in a subject in need thereof, comprising administering to the subject a therapeutically effective amount of a mesylate salt of Compound (I) or one of the mesylate crystalline forms disclosed herein, or administering a crystalline polymorph of Compound (I) free base as disclosed herein. In some embodiments, the cancer is bladder cancer, prostate cancer, breast cancer, cervical cancer, colorectal cancer, endometrial cancer, gastric cancer, glioblastoma, head and neck cancer, lung cancer, urothelial cancer, sinonasal cancer, or non-small cell lung cancer. In some embodiments, the cancer in the subject characterized by an EGFR mutation.

The present disclosure also provides a use of the salt or freebase of Compound (I) of the disclosure or a pharmaceutical composition thereof comprising the same for the treatment of any of the disease recited in the previous paragraph. In one embodiment, provided is the salt or freebase of the disclosure or a pharmaceutical composition thereof comprising the same for use in any of the method of the disclosure described herein. In another embodiment, provided is use of the salt or freebase of the disclosure or a pharmaceutical composition thereof comprising the same for the manufacture of a medicament for any of the method of the disclosure described.

### BRIEF DESCRIPTION OF THE DRAWINGS

FIG. 1A shows the X-ray Powder Diffraction (XRPD) pattern of Form A of a mesylate salt of Compound (I), wherein the molar ratio between Compound (I) and methanesulfonic acid is 1:1.

FIG. 1B shows the Thermogravimetric Analysis (TGA) and Differential Scanning Calorimetry Analysis (DSC) thermograms of Form A of a mesylate salt of Compound (I), wherein the molar ratio between Compound (I) and methanesulfonic acid is 1:1.

FIG. 2A shows the X-ray Powder Diffraction (XRPD) pattern of the Form B of a mesylate salt of Compound (I), wherein the molar ratio between Compound (I) and methanesulfonic acid is 1:1.

FIG. 2B shows the Thermogravimetric Analysis (TGA) and Differential Scanning Calorimetry Analysis (DSC) thermograms of the Form B of a mesylate salt of Compound (I), wherein the molar ratio between Compound (I) and methanesulfonic acid is 1:1.

FIG. 3A shows the X-ray Powder Diffraction (XRPD) pattern of the Form H of a mesylate salt of Compound (I), wherein the molar ratio between Compound (I) and methanesulfonic acid is 1:1.

FIG. 3B shows the Thermogravimetric Analysis (TGA) and Differential Scanning Calorimetry Analysis (DSC) thermograms of the Form H of a mesylate salt of Compound (I), wherein the molar ratio between Compound (I) and methanesulfonic acid is 1:1.

FIG. 4A shows the X-ray Powder Diffraction (XRPD) pattern of Form I of a mesylate salt of Compound (I), wherein the molar ratio between Compound (I) and methane sulfonic acid is 1:1.

FIG. 4B shows the Thermogravimetric Analysis (TGA) and Differential Scanning Calorimetry Analysis (DSC) thermograms of the Form I of a mesylate salt of Compound (I), wherein the molar ratio between Compound (I) and methanesulfonic acid is 1:1.

FIG. 5A shows the X-ray Powder Diffraction (XRPD) pattern of the Crystalline Form A of Compound (I) free base.

FIG. 5B shows the Thermogravimetric Analysis (TGA) and Differential Scanning Calorimetry Analysis (DSC) thermograms of the Crystalline Form A of Compound (I) free base.

FIG. 6A shows the X-ray Powder Diffraction (XRPD) pattern of the Crystalline Form B of Compound (I) free base.

FIG. 6B shows the Differential Scanning Calorimetry Analysis (DSC) thermograms of the Crystalline Form B of Compound (I) free base.

FIG. 7 shows mean concentration in the plasma of male beagle dogs following a single 30 mg/kg PO dose of compound (I) free base or mesylate salt.

### DETAILED DESCRIPTION

The present disclosure is directed to mesylate salts of Compound (I) having different solid forms and to free base crystalline forms of Compound (I) having different solid forms.

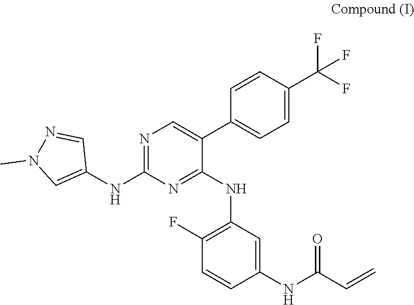 [SMILES: C=CC(=O)Nc1ccc(F)c(Nc2nc(Nc3cnn(C)c3)ncc2-c2ccc(C(F)(F)F)cc2)c1]

As used herein, “crystalline” refers to a solid having a crystal structure wherein the individual molecules have a highly homogeneous regular three dimensional configuration.

In some embodiments, for the crystalline forms of Compound (I) salt or free base disclosed herein, at least a particular percentage by weight of the Compound (I) salt or free base is in a particular crystalline form. Particular weight percentages include 85%, 86%, 87%, 88%, 89%, 90%, 91%, 92%, 93%, 94%, 95%, 96%, 97%, 98%, 99%, 99.5%, 99.9%, or a weight percentage of 70%-75%, 75%-80%, 80%-85%, 85%-90%, 90%-95%, 95%-100%, 70-80%, 80-90%, 90-100% by weight of the Compound (I) salt or free base is in a particular crystalline form. It is to be understood that all values and ranges between these values and ranges are meant to be encompassed by the present disclosure.

When the crystalline Compound (I) salt or free base is defined as a specified percentage of one particular crystal form of the Compound (I) salt or free base, the remainder is made up of amorphous form and/or crystal forms other than the one or more particular forms that are specified.

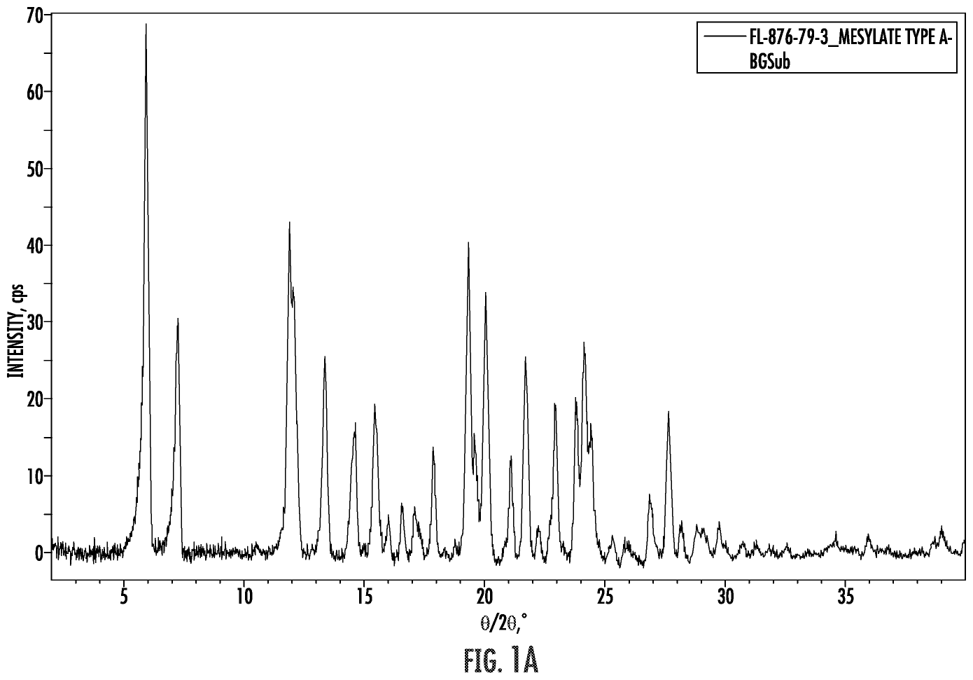

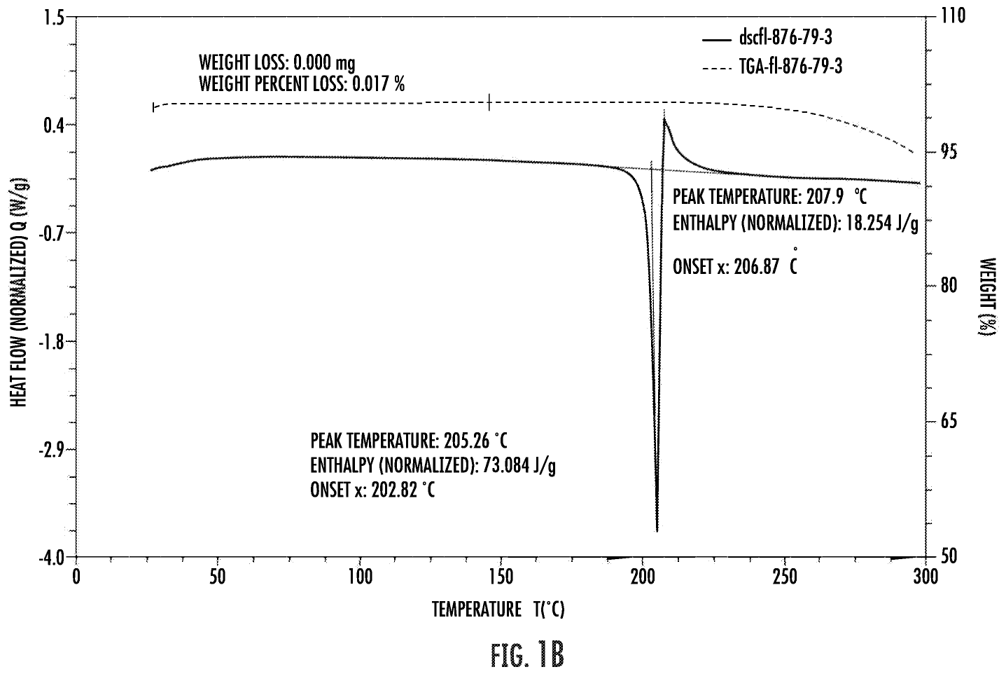

The crystalline Compound (I) salts disclosed herein exhibit strong, unique XRPD patterns with sharp peaks corresponding to angular peak positions in 2θ and a flat baseline, indicative of a highly crystalline material (e.g., see FIG. 1A).

As used herein, an X-ray powder diffractogram is “substantially similar to that in [a particular] Figure” when at least 90%, such as at least 95%, at least 98%, or at least 99%, of the signals in the two diffractograms are the same±0.2° 2θ. In determining “substantial similarity,” one of ordinary skill in the art will understand that there may be variation in the intensities and/or signal positions in XRPD diffractograms even for the same crystalline form. Thus, those of ordinary skill in the art will understand that the signal maximum values in XRPD diffractograms (in degrees two-theta (° 20) referred to herein) generally mean that value reported±0.2 degrees 2θ of the reported value, an art-recognized variance discussed above. In some embodiments, when a crystalline form is characterized by XRPD peaks with specific values, one of ordinary skill in the art will understand that the peak values can be ±0.4, ±0.3, ±0.2 or 0.1 degrees 2θ of the reported values, unless specified otherwise.

### Mesylate Salts of Compound (I)

In one aspect, the present disclosure provides a mesylate salt of Compound (I).

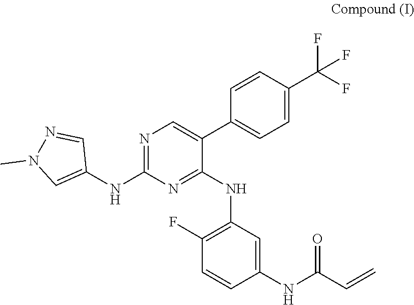 [SMILES: C=CC(=O)Nc1ccc(F)c(Nc2nc(Nc3cnn(C)c3)ncc2-c2ccc(C(F)(F)F)cc2)c1]

wherein the molar ratio between Compound (I) and methanesulfonic acid is about 1:1.

In some embodiments, the mesylate salt is crystalline mesylate salt. In some embodiments, the mesylate salt is in a single crystalline form.

In some embodiments, the mesylate salt is unsolvated. In other embodiments, the mesylate salt is solvated.

A mesylate salt of Compound (I) with 1:1 molar ratio between Compound (I) and methanesulfonic acid can be readily prepared by mixing Compound (I) free base with about 1-1.1 equivalent of methanesulfonic acid in a suitable solvent (e.g., acetone, THF). The mixture can be heated in order to get a desired yield.

### Mesylate Salt of Form A

In some embodiments, the present disclosure provides a crystalline Form A of mesylate salt of Compound (I), wherein the molar ratio between Compound (I) and methanesulfonic acid is 1:1. In some embodiments, crystalline Form A of mesylate salt of Compound (I) is an anhydrate.

The XRPD pattern and peaks are shown in FIG. 1A, and the Thermogravimetric Analysis (TGA) and Differential Scanning Calorimetry Analysis (DSC) thermograms are shown in FIG. 1B.

TABLE 1
Peak list for Compound (I) mesylate Form A

| 2-theta | d-Spacing | Relative |
|---|---|---|
| deg | (Å) | Intensity |
| 5.94 | 14.86 | 100 |
| 7.22 | 12.23 | 42 |
| 11.86 | 7.45 | 40 |
| 12.06 | 7.33 | 44 |
| 13.35 | 6.63 | 32 |
| 14.60 | 6.06 | 24 |
| 15.44 | 5.74 | 28 |
| 16.55 | 5.35 | 7 |
| 17.10 | 5.18 | 10 |
| 17.85 | 4.97 | 14 |
| 19.32 | 4.59 | 46 |
| 19.61 | 4.52 | 15 |
| 20.05 | 4.43 | 48 |
| 21.08 | 4.21 | 13 |
| 21.72 | 4.09 | 33 |
| 22.94 | 3.87 | 26 |
| 23.80 | 3.74 | 24 |
| 24.14 | 3.68 | 33 |
| 24.42 | 3.64 | 18 |
| 26.86 | 3.32 | 9 |
| 27.64 | 3.23 | 23 |
| 28.15 | 3.17 | 6 |
| 28.93 | 3.08 | 11 |
| 34.62 | 2.59 | 5 |

In Table 1 only those peaks with a relative intensity of five or greater compared to the absolute intensity (I. in cps°) of the most intense peak are reported.

TABLE 2
Condensed peak list #1 for Compound (I) mesylate Form A

| 2-theta | d-Spacing | Relative |
|---|---|---|
| (deg) | (Å) | Intensity |
| 5.94 | 14.86 | 100 |
| 7.22 | 12.23 | 42 |
| 11.86 | 7.45 | 40 |
| 12.06 | 7.33 | 44 |
| 13.35 | 6.63 | 32 |
| 14.60 | 6.06 | 24 |
| 15.44 | 5.74 | 28 |
| 19.32 | 4.59 | 46 |
| 20.05 | 4.43 | 48 |
| 21.72 | 4.09 | 33 |
| 22.94 | 3.87 | 26 |
| 23.80 | 3.74 | 24 |
| 24.14 | 3.68 | 33 |
| 27.64 | 3.23 | 23 |

TABLE 3
Condensed peak list #2 for Compound (I) mesylate Form A

| 2-theta | d-Spacing | Relative |
|---|---|---|
| (deg) | (Å) | Intensity |
| 5.94 | 14.86 | 100 |
| 7.22 | 12.23 | 42 |
| 11.86 | 7.45 | 40 |
| 12.06 | 7.33 | 44 |
| 13.35 | 6.63 | 32 |
| 19.32 | 4.59 | 46 |
| 20.05 | 4.43 | 48 |
| 21.72 | 4.09 | 33 |
| 24.14 | 3.68 | 33 |
| 27.64 | 3.23 | 23 |

TABLE 4
Condensed peak list #3 for Compound (I) mesylate Form A

| 2-theta | d-Spacing | Relative |
|---|---|---|
| (deg) | (Å) | Intensity |
| 5.94 | 14.86 | 100 |
| 7.22 | 12.23 | 42 |
| 11.86 | 7.45 | 40 |
| 12.06 | 7.33 | 44 |
| 19.32 | 4.59 | 46 |
| 20.05 | 4.43 | 48 |

In some embodiments, the mesylate salt of Form A is characterized by an X-ray powder diffraction pattern comprising at least three, at least four, or at least five peaks selected from 5.9°, 7.2°, 11.9°, 12.1°, 19.3° and 20.1°±0.2° in 2θ.

In some embodiments, the mesylate salt of Form A is characterized by an X-ray powder diffraction pattern comprising peaks at 5.9°, 7.2°, 11.9°, 12.1°, 19.3° and 20.1°±0.2° in 2θ.

In some embodiments, the mesylate salt of Form A is characterized by an X-ray powder diffraction pattern comprising at least three, at least four, at least five, at least six, at least seven, at least eight or at least nine peaks at 5.9°, 7.2°, 11.9°, 12.1°, 13.4°, 19.3°, 20.1°, 21.7°, 24.1° and 27.6°±0.2° in 2θ.

In some embodiments, the mesylate salt of Form A is characterized by an X-ray powder diffraction pattern comprising peaks at 5.9°, 7.2°, 11.9°, 12.1°, 13.4°, 19.3°, 20.1°, 21.7°, 24.10, and 27.6°±0.2° in 2θ.

In some embodiments, the mesylate salt of Form A is characterized by an X-ray powder diffraction pattern comprising at least three, at least four, at least five, at least six, at least seven, at least eight, at least nine, at least ten, at least eleven, at least twelve, or at least thirteen peaks at 5.9°, 7.2°, 11.9°, 12.1°, 13.4°, 14.6°, 15.4°, 19.3°, 20.1°, 21.7°, 22.9°, 23.8°, 24.1° and 27.6°±0.20 in 2θ.

In some embodiments, the mesylate salt of Form A is characterized by an X-ray powder diffraction pattern comprising peaks at 5.9°, 7.2°, 11.9°, 12.1°, 13.4°, 14.6°, 15.4°, 19.3°, 20.1°, 21.7°, 22.9°, 23.8°, 24.1° and 27.6°±0.2° in 2θ.

In some embodiments, the mesylate salt of Form A is characterized by an X-ray powder diffraction pattern substantially similar to FIG. 1A.

In some embodiments, the mesylate salt of Form A is characterized by a differential scanning calorimeter (DSC) thermogram comprising two endothermic and exothermic events; an endotherm with an onset of 202.8° C.±2° C., and an exotherm with an onset of 206.9° C.±2° C. In some embodiments, the mesylate salt of Form A is characterized by a differential scanning calorimeter (DSC) thermogram substantially similar to that in FIG. 1B.

In some embodiments, the mesylate salt of Form A is characterized by a thermogravimetric analysis (TGA) substantially similar to that in FIG. 1B.

### Mesylate Salt of Form B

In some embodiments, the present disclosure provides a crystalline Form B of mesylate salt of Compound (I), wherein the molar ratio between Compound (I) and methanesulfonic acid is 1:1. In some embodiments, crystalline Form B of mesylate salt of Compound (I) is an anhydrate.

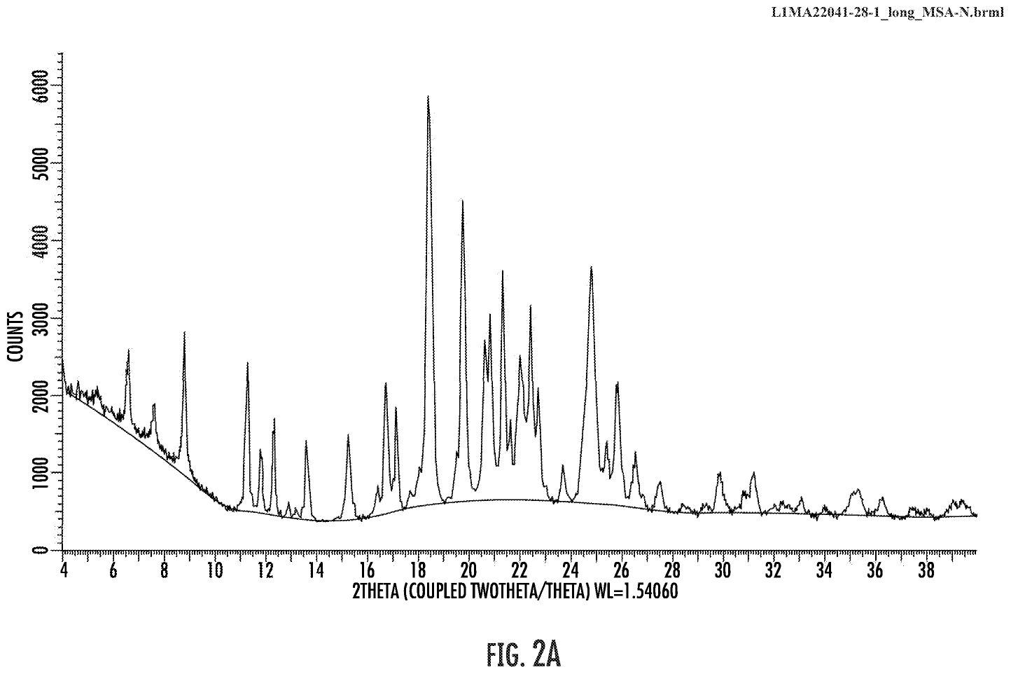

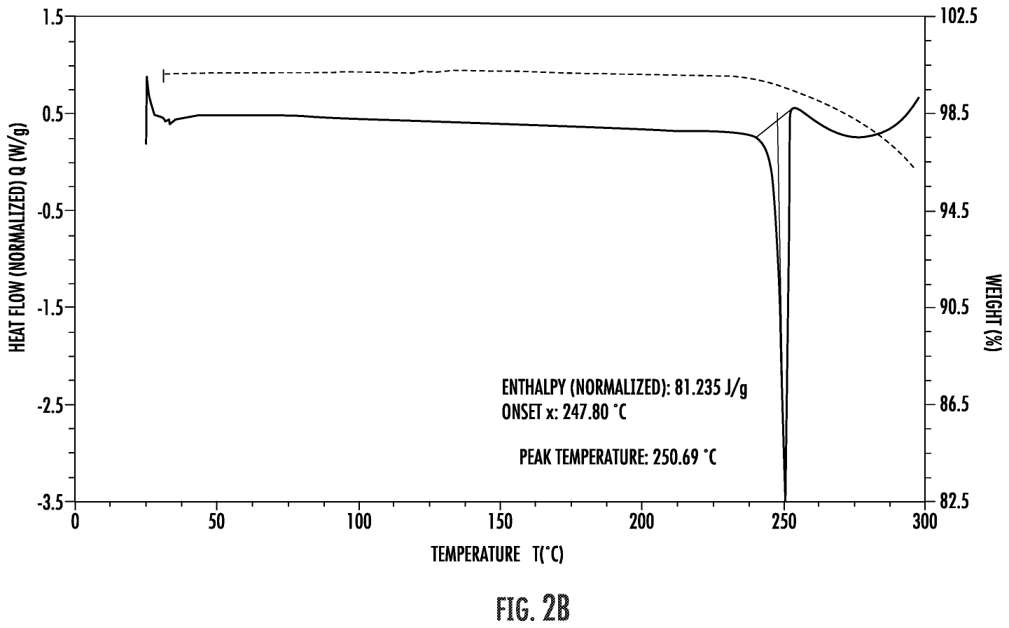

The XRPD pattern and peaks are shown in FIG. 2A, and the Thermogravimetric Analysis (TGA) and Differential Scanning Calorimetry Analysis (DSC) thermograms are shown in FIG. 2B.

TABLE 5a
Peak list for Compound (I) mesylate Form B

| 2-theta | d-Spacing | Relative |
|---|---|---|
| (deg) | (Å) | Intensity |
| 6.66 | 13.26 | 13 |
| 7.69 | 11.49 | 12 |
| 8.92 | 9.91 | 24 |
| 11.30 | 7.82 | 29 |
| 11.85 | 7.47 | 11 |
| 12.36 | 7.16 | 12 |
| 13.00 | 6.80 | 6 |
| 13.72 | 6.45 | 13 |
| 15.35 | 5.77 | 17 |
| 16.78 | 5.28 | 21 |
| 17.19 | 5.15 | 9 |
| 18.50 | 4.79 | 100 |
| 19.83 | 4.47 | 41 |
| 20.84 | 4.26 | 48 |
| 21.40 | 4.15 | 27 |
| 22.42 | 3.96 | 54 |
| 24.91 | 3.57 | 52 |
| 25.90 | 3.44 | 31 |
| 26.75 | 3.33 | 5 |
| 29.99 | 2.98 | 5 |
| 31.12 | 2.87 | 9 |
| 35.24 | 2.54 | 7 |

TABLE 5b
Peak list for Compound (I) mesylate Form B

| 2-theta | d-spacing | Relative |
|---|---|---|
| (deg.) | (ang.) | intensity |
| 5.35 | 16.49 | 5 |
| 6.59 | 13.41 | 20 |
| 7.59 | 11.64 | 11 |
| 8.80 | 10.04 | 34 |
| 11.28 | 7.84 | 36 |
| 11.80 | 7.49 | 15 |
| 12.30 | 7.19 | 24 |
| 13.58 | 6.51 | 19 |
| 15.24 | 5.81 | 20 |
| 15.43 | 5.74 | 5 |
| 16.37 | 5.41 | 7 |
| 16.73 | 5.30 | 32 |
| 17.12 | 5.18 | 25 |
| 18.02 | 4.92 | 9 |
| 18.40 | 4.82 | 100 |
| 19.48 | 4.55 | 13 |
| 19.74 | 4.49 | 73 |
| 20.63 | 4.30 | 39 |
| 20.82 | 4.26 | 46 |
| 21.32 | 4.16 | 56 |
| 21.60 | 4.11 | 19 |
| 22.01 | 4.04 | 35 |
| 22.41 | 3.96 | 47 |
| 22.70 | 3.91 | 27 |
| 23.69 | 3.75 | 9 |
| 24.50 | 3.63 | 21 |
| 24.79 | 3.59 | 58 |
| 25.40 | 3.50 | 15 |
| 25.80 | 3.45 | 29 |
| 25.74 | 3.46 | 22 |
| 26.53 | 3.36 | 13 |
| 27.51 | 3.24 | 7 |
| 29.82 | 2.99 | 9 |
| 30.84 | 2.90 | 5 |
| 31.21 | 2.86 | 10 |
| 35.04 | 2.56 | 6 |
| 35.31 | 2.54 | 7 |
| 36.25 | 2.48 | 5 |

In Tables 5a and 5b, only those peaks with a relative intensity of five or greater compared to the absolute intensity (I. in cps°) of the most intense peak are reported. Tables 5a and 5b are XRPD peaks obtained from two different batches of crystalline Form B of mesylate salt of Compound (I). Tables 6a and 7a are condensed peak lists selected from those in Table 5a and Tables 6b and 7b are condensed peak lists selected from those in Table 5b.

TABLE 6a
Condensed peak list #1 for Compound (I) mesylate Form B

| 2-theta | d-Spacing | Relative |
|---|---|---|
| (deg) | (Å) | Intensity |
| 8.92 | 9.91 | 24 |
| 11.30 | 7.82 | 29 |
| 18.50 | 4.79 | 100 |
| 19.83 | 4.47 | 41 |
| 20.84 | 4.26 | 48 |
| 21.40 | 4.15 | 27 |
| 22.42 | 3.96 | 54 |
| 24.91 | 3.57 | 52 |
| 25.90 | 3.44 | 31 |

TABLE 6b
Condensed peak list #1 for Compound (I) mesylate Form B

| | d-spacing | Relative |
|---|---|---|
| 2-theta (deg.) | (ang.) | intensity |
| 18.40 | 4.82 | 100 |
| 19.74 | 4.49 | 73 |
| 24.79 | 3.59 | 58 |
| 21.32 | 4.16 | 56 |
| 22.41 | 3.96 | 47 |
| 20.82 | 4.26 | 46 |
| 20.63 | 4.30 | 39 |
| 11.28 | 7.84 | 36 |
| 22.01 | 4.04 | 35 |
| 8.80 | 10.04 | 34 |

TABLE 7a
Condensed peak list #2 for Compound (I) mesylate Form B

| 2-theta | d-Spacing | Relative |
|---|---|---|
| deg | (Å) | Intensity |
| 18.50 | 4.79 | 100 |
| 19.83 | 4.47 | 41 |
| 20.84 | 4.26 | 48 |
| 22.42 | 3.96 | 54 |
| 24.91 | 3.57 | 52 |

TABLE 7b
Condensed peak list #2 for Compound (I) mesylate Form B

| 2-theta | d-spacing | Relative |
|---|---|---|
| (deg.) | ang. | intensity |
| 18.40 | 4.82 | 100 |
| 19.74 | 4.49 | 73 |
| 24.79 | 3.59 | 58 |
| 21.32 | 4.16 | 56 |
| 22.41 | 3.96 | 47 |

In some embodiments, the mesylate salt of Form B is characterized by an X-ray powder diffraction pattern comprising at least three or at least four peaks selected from 18.5°, 19.8°, 20.8°, 22.4° and 24.9° in 2θ. In some embodiments, the mesylate salt of Form B is characterized by an X-ray powder diffraction pattern comprising peaks at 18.5°, 19.8°, 20.8°, 22.4° and 24.9° in 2θ

In some embodiments, the mesylate salt of Form B is characterized by an X-ray powder diffraction pattern comprising at least three or at least four peaks selected from 18.5°, 19.8°, 20.8°, 22.4° and 24.9°±0.2° in 2θ.

In some embodiments, the mesylate salt of Form B is characterized by an X-ray powder diffraction pattern comprising peaks at 18.5°, 19.8°, 20.8°, 22.4° and 24.9°±0.2° in 2θ.

In some embodiments, the mesylate salt of Form B is characterized by an X-ray powder diffraction pattern comprising at least three, at least four, at least five, at least six, at least seven, or at least eight peaks at 8.9°, 11.3°, 18.5°, 19.8°, 20.8°, 21.4°, 22.4°, 24.9°, and 25.9°±0.2° in 2θ.

In some embodiments, the mesylate salt of Form B is characterized by an X-ray powder diffraction pattern comprising peaks at 8.9°, 11.3°, 18.5°, 19.8°, 20.8°, 21.4°, 22.4°, 24.9°, and 25.9°±0.2° in 2θ.

In some embodiments, the mesylate salt of Form B is characterized by an X-ray powder diffraction pattern comprising at least three or at least four peaks selected from 18.4°, 19.7°. 21.3°, 22.4°, 24.8° in 2θ. In some embodiments, the mesylate salt of Form B is characterized by an X-ray powder diffraction pattern comprising peaks at 18.4°, 19.7°. 21.3°, 22.4°, 24.8° in 2θ.

In some embodiments, the mesylate salt of Form B is characterized by an X-ray powder diffraction pattern comprising at least three or at least four peaks selected from 18.4°, 19.7°. 21.3°, 22.4°, 24.8°±0.2° in 2θ.

In some embodiments, the mesylate salt of Form B is characterized by an X-ray powder diffraction pattern comprising peaks at 18.4°, 19.7°. 21.3°, 22.4°, 24.8°±0.2° in 2θ.

In some embodiments, the mesylate salt of Form B is characterized by an X-ray powder diffraction pattern comprising at least three, at least four, at least five, at least six, at least seven, at least eight, or at least nine peaks at 8.8°, 11.3°, 18.4°, 19.7°, 20.6°, 20.8°, 21.3°, 22.0°, 22.4° and 24.8°±0.2° in 2θ.

In some embodiments, the mesylate salt of Form B is characterized by an X-ray powder diffraction pattern comprising peaks at 8.8°, 11.3°, 18.4°, 19.7°, 20.6°, 20.8°, 21.3°, 22.0°, 22.4° and 24.8°±0.2° in 2θ.

In some embodiments, the mesylate salt of Form B is characterized by an X-ray powder diffraction pattern comprising peaks at 6.7°, 7.7°, 8.9°, 11.3°, 11.9°, 12.4°, 13.0°, 13.7°, 15.4°, 16.8°, 17.2°, 18.5°, 19.8°, 20.9°, 21.4°, 22.4°, 24.9°, 25.9°, 26.8°, 30.0°, 31.1°, and 35.2°±0.2° in 20.

In some embodiments, the mesylate salt of Form B is characterized by an X-ray powder diffraction pattern comprising peaks at 5.4°, 6.6°, 7.6°, 8.8°, 11.3°, 11.8°, 12.3°, 13.6°, 15.2°, 15.4°, 16.4°, 16.7°, 17.1°, 18.0°, 18.4°, 19.5°, 19.7°, 20.6°, 20.8°, 21.3°, 21.6°, 22.0°, 22.4°, 22.7°, 23.7°, 24.5°, 24.8°, 25.4°, 25.7°, 25.8°, 26.5°, 27.5°, 29.8°, 30.8°, 31.2°, 35.0°, 35.3° and 36.3°±0.2° in 2θ. In some embodiments, the mesylate salt of Form B is characterized by an X-ray powder diffraction pattern substantially similar to FIG. 2A.

In some embodiments, the mesylate salt of Form B is characterized by a differential scanning calorimeter (DSC) thermogram comprising an endotherm onset at 247.8° C.±2° C. In some embodiments, the mesylate salt of Form B is characterized by a differential scanning calorimeter (DSC) thermogram substantially similar to that in FIG. 2B.

In some embodiments, the mesylate salt of Form B is characterized by a thermogravimetric analysis (TGA) substantially similar to that in FIG. 2B.

In some embodiments, the mesylate salt of Form B is characterized by a differential scanning calorimeter (DSC) thermogram comprising an endotherm onset at 250.1° C.±2° C. In some embodiments, the DSC thermogram further comprises an exotherm onset at 254.1° C.±2° C. In some embodiments, the DSC was performed using a hermetic aluminum pan with pinhole.

In some embodiments, the mesylate salt of Form B is prepared by crystallization from acetone. In some embodiments, the mesylate salt of Form B is prepared by mixing Compound (I) free base and methanesulfonic acid (e.g., in an amount of 1-1.5, 1-1.4, 1-1.2 or 1-1.1 molar equivalent to Compound (I)) in acetone at an elevated temperature (e.g., between 40° C. and 70° C., between 40° C. and 65° C., between 45° C. and 55° C. or between 50° C. and 60° C., etc.) followed by cooling the mixture to form the mesylate salt of Form B. In some embodiments, the mixture is cooled to an ambient or lower temperature, e.g., between 5° C. and 25° C., between 5° C. and 15° C., between 15° C. and 25° C., etc. In some embodiments, the mesylate salt of Form B is prepared by (i) mixing Compound (I) free base with a small portion of the methanesulfonic acid (e.g., 0.1-0.5 molar equivalent to compound (I)) at an elevated temperature (e.g., between 40° C. and 70° C., between 40° C. and 65° C., between 45° C. and 55° C. or between 50° C. and 60° C., etc.); (ii) adding seed crystal of mesylate form B; (iii) adding the remainder of methanesulfonic acid (e.g., 0.6-1.0 molar equivalent); and (iv) cooling the mixture to form the mesylate salt of Form B. In some embodiments, the mixture is cooled to an ambient or lower temperature, e.g., between 5° C. and 25° C., between 5° C. and 15° C., between 15° C. and 25° C., etc. In some embodiments, the mesylate salt of Form B formed from the methods described above is washed with acetone and dried at an elevated temperature (e.g., between 40° C. and 70° C., between 40° C. and 65° C., between 45° C. and 55° C. or between 50° C. and 60° C., etc.). In some embodiments, the methods of preparing the mesylate Form B described above can be carried out in a mixture of acetone and small amount of water ((e.g., 1-5%) instead of acetone.

### Mesylate Salt of Form H

In some embodiments, the present disclosure provides a crystalline Form H of mesylate salt of Compound (I), wherein the molar ratio between Compound (I) and methanesulfonic acid is 1:1. In some embodiments, crystalline Form H of mesylate salt of Compound (I) is an anhydrate.

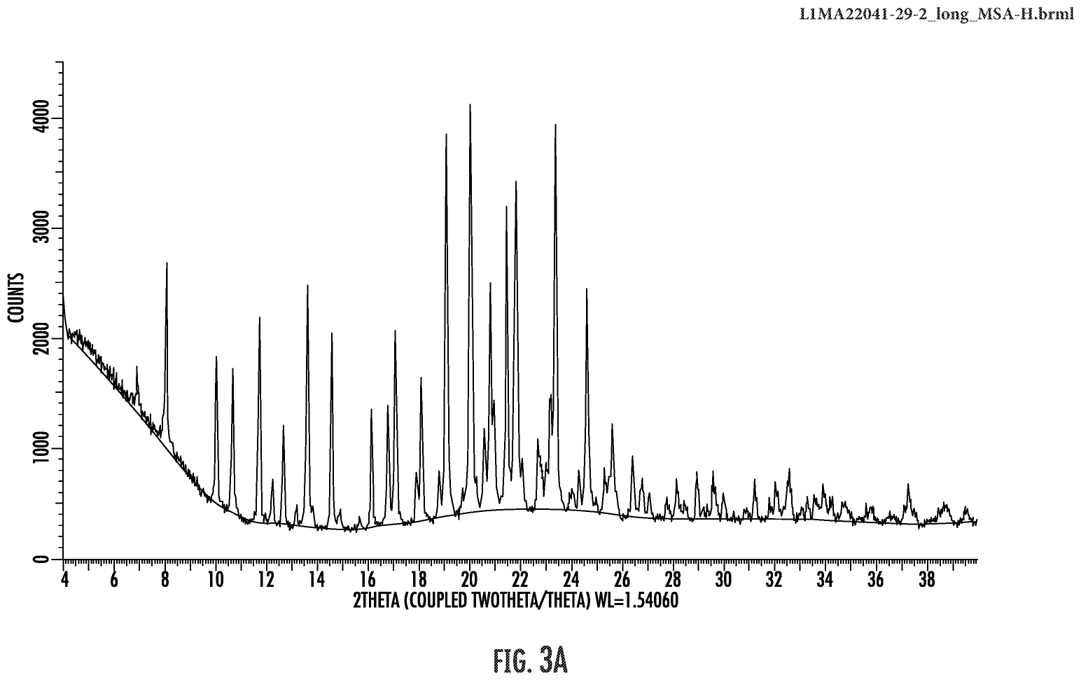

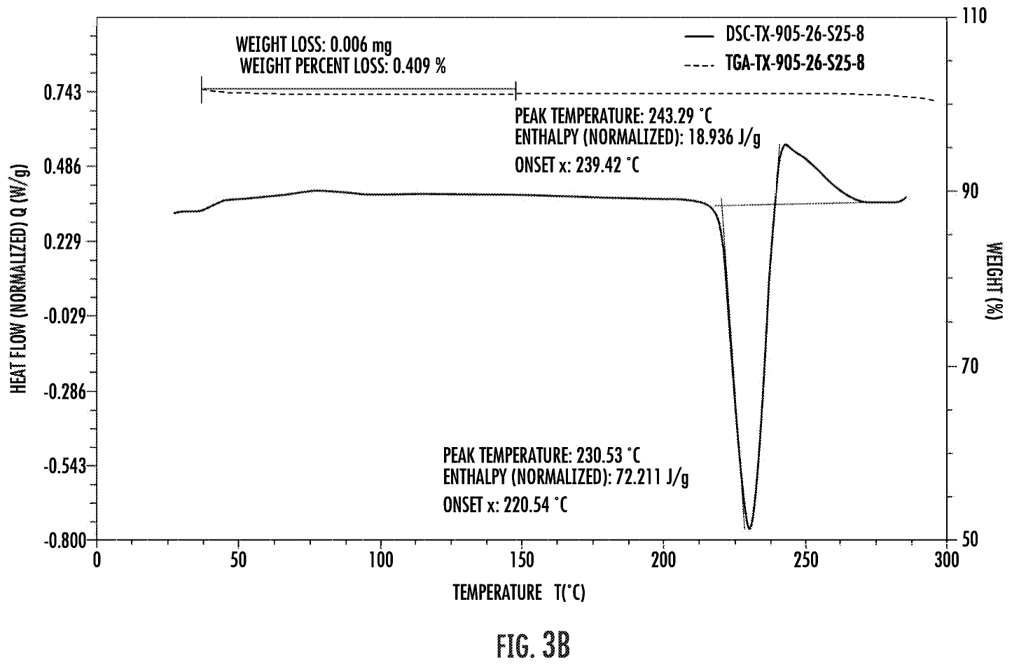

The XRPD pattern and peaks are shown in FIG. 3A, and the Thermogravimetric Analysis (TGA) and Differential Scanning Calorimetry Analysis (DSC) thermograms are shown in FIG. 3B.

TABLE 8a
Peak list for Compound (I) mesylate Form H

| 2-theta | d-Spacing | Relative |
|---|---|---|
| (deg) | (Å) | Intensity |
| 6.95 | 12.71 | 15 |
| 8.09 | 10.92 | 41 |
| 10.07 | 8.78 | 40 |
| 10.72 | 8.24 | 37 |
| 11.74 | 7.53 | 40 |
| 12.28 | 7.20 | 13 |
| 12.70 | 6.97 | 29 |
| 13.20 | 6.70 | 6 |
| 13.65 | 6.48 | 59 |
| 13.85 | 6.39 | 5 |
| 14.59 | 6.07 | 30 |
| 14.92 | 5.93 | 6 |
| 16.15 | 5.48 | 14 |
| 16.81 | 5.27 | 29 |
| 17.06 | 5.19 | 26 |
| 17.92 | 4.95 | 8 |
| 18.09 | 4.90 | 21 |
| 18.81 | 4.72 | 9 |
| 19.11 | 4.64 | 51 |
| 19.16 | 4.63 | 19 |
| 20.04 | 4.43 | 100 |
| 20.61 | 4.31 | 12 |
| 20.81 | 4.27 | 44 |
| 20.88 | 4.25 | 5 |
| 20.95 | 4.24 | 19 |
| 21.48 | 4.13 | 55 |
| 21.85 | 4.06 | 71 |
| 22.08 | 4.02 | 8 |
| 22.71 | 3.91 | 10 |
| 22.85 | 3.89 | 9 |
| 23.20 | 3.83 | 23 |
| 23.40 | 3.80 | 59 |
| 23.46 | 3.79 | 19 |
| 23.94 | 3.71 | 6 |
| 24.65 | 3.61 | 30 |
| 24.72 | 3.60 | 9 |
| 25.64 | 3.47 | 25 |
| 26.45 | 3.37 | 12 |
| 26.79 | 3.33 | 8 |
| 27.76 | 3.21 | 5 |
| 28.16 | 3.17 | 8 |
| 28.98 | 3.08 | 8 |
| 29.68 | 3.01 | 5 |
| 30.03 | 2.97 | 5 |
| 32.11 | 2.78 | 7 |
| 32.57 | 2.75 | 8 |
| 33.95 | 2.64 | 5 |
| 37.31 | 2.41 | 6 |
| 38.81 | 2.32 | 5 |

TABLE 8b
Peak list for Compound (I) mesylate Form H

| 2-theta | d-spacing | Relative |
|---|---|---|
| (deg.) | (ang.) | intensity |
| 6.92 | 12.76 | 11 |
| 8.07 | 10.94 | 45 |
| 10.05 | 8.79 | 36 |
| 10.70 | 8.26 | 36 |
| 11.74 | 7.53 | 50 |
| 12.26 | 7.22 | 11 |
| 12.69 | 6.97 | 24 |
| 13.18 | 6.71 | 5 |
| 13.63 | 6.49 | 60 |
| 13.85 | 6.39 | 5 |
| 14.59 | 6.07 | 48 |
| 14.89 | 5.94 | 5 |
| 16.16 | 5.48 | 29 |
| 16.80 | 5.27 | 29 |
| 17.08 | 5.19 | 48 |
| 17.93 | 4.94 | 12 |
| 18.11 | 4.89 | 35 |
| 18.82 | 4.71 | 11 |
| 19.09 | 4.65 | 95 |
| 19.72 | 4.50 | 6 |
| 20.02 | 4.43 | 100 |
| 20.59 | 4.31 | 19 |
| 20.82 | 4.26 | 56 |
| 20.95 | 4.24 | 26 |
| 21.47 | 4.13 | 75 |
| 21.85 | 4.06 | 82 |
| 22.07 | 4.02 | 13 |
| 22.76 | 3.90 | 13 |
| 23.19 | 3.83 | 27 |
| 23.38 | 3.80 | 95 |
| 24.06 | 3.70 | 5 |
| 24.31 | 3.66 | 10 |
| 24.63 | 3.61 | 53 |
| 25.38 | 3.51 | 7 |
| 25.63 | 3.47 | 22 |
| 26.42 | 3.37 | 15 |
| 26.78 | 3.33 | 10 |
| 27.08 | 3.29 | 6 |
| 27.75 | 3.21 | 5 |
| 28.16 | 3.17 | 10 |
| 28.96 | 3.08 | 12 |
| 29.63 | 3.01 | 6 |
| 30.00 | 2.98 | 7 |
| 31.23 | 2.86 | 10 |
| 31.81 | 2.81 | 5 |
| 32.05 | 2.79 | 8 |
| 32.60 | 2.74 | 13 |
| 33.30 | 2.69 | 5 |
| 33.96 | 2.64 | 6 |
| 34.28 | 2.61 | 6 |
| 34.80 | 2.58 | 5 |
| 35.62 | 2.52 | 5 |
| 37.28 | 2.41 | 10 |
| 38.72 | 2.32 | 5 |

In Tables 8a and 8b only those peaks with a relative intensity of five or greater compared to the absolute intensity (I. in cps°) of the most intense peak are reported. Tables 8a and 8b are XRPD peaks obtained from two different batches of crystalline Form H of mesylate salt of Compound (I). Tables 9a and 10a are condensed peak lists selected from those in Table 8a and Tables 9b and 10b are condensed peak lists selected from those in Table 8b.

TABLE 9a
Condensed peak list #1 for Compound (I) mesylate Form H

| 2-theta | d-Spacing | Relative |
|---|---|---|
| (deg) | (Å) | Intensity |
| 8.09 | 10.92 | 41 |
| 10.07 | 8.78 | 40 |
| 10.72 | 8.24 | 37 |
| 11.74 | 7.53 | 40 |
| 13.65 | 6.48 | 59 |
| 14.59 | 6.07 | 30 |
| 19.11 | 4.64 | 51 |
| 20.04 | 4.43 | 100 |
| 20.81 | 4.27 | 44 |
| 21.48 | 4.13 | 55 |
| 21.85 | 4.06 | 71 |
| 23.40 | 3.80 | 59 |
| 24.65 | 3.61 | 30 |

TABLE 9b
Condensed peak list #1 for Compound (I) mesylate Form H

| 2-theta | d-spacing | Relative |
|---|---|---|
| (deg.) | (ang.) | intensity |
| 20.02 | 4.43 | 100 |
| 19.09 | 4.65 | 95 |
| 23.38 | 3.80 | 95 |
| 21.85 | 4.06 | 82 |
| 21.47 | 4.13 | 75 |
| 13.63 | 6.49 | 60 |
| 20.82 | 4.26 | 56 |
| 24.63 | 3.61 | 53 |
| 11.74 | 7.53 | 50 |
| 14.59 | 6.07 | 48 |

TABLE 10a
Condensed peak list #2 for Compound (I) mesylate Form H

| 2-theta | d-Spacing | Relative |
|---|---|---|
| deg | (Å) | Intensity |
| 8.09 | 10.92 | 41 |
| 10.07 | 8.78 | 40 |
| 11.74 | 7.53 | 40 |
| 13.65 | 6.48 | 59 |
| 19.11 | 4.64 | 51 |
| 20.04 | 4.43 | 100 |
| 20.81 | 4.27 | 44 |
| 21.48 | 4.13 | 55 |
| 21.85 | 4.06 | 71 |
| 23.40 | 3.80 | 59 |

TABLE 10b
Condensed peak list #2 for Compound (I) mesylate Form H

| 2-theta | d-spacing | Relative |
|---|---|---|
| (deg.) | (ang.) | intensity |
| 20.02 | 4.43 | 100 |
| 19.09 | 4.65 | 95 |
| 23.38 | 3.80 | 95 |
| 21.85 | 4.06 | 82 |
| 21.47 | 4.13 | 75 |

In some embodiments, the mesylate salt of Form H is characterized by an X-ray powder diffraction pattern comprising at least three, at least four or at least five peaks selected from 13.7°, 19.1°, 20.0°, 21.5°, 21.9°, and 23.40 in 2θ. In some embodiments, the mesylate salt of Form H is characterized by an X-ray powder diffraction pattern comprising peaks at 13.7°, 19.1°, 20.0°, 21.5°, 21.9°, and 23.4° in 2θ

In some embodiments, the mesylate salt of Form H is characterized by an X-ray powder diffraction pattern comprising at least three, at least four or at least five peaks selected from 13.7°, 19.1°, 20.0°, 21.5°, 21.9°, and 23.4°±0.2° in 2θ.

In some embodiments, the mesylate salt of Form H is characterized by an X-ray powder diffraction pattern comprising peaks at 13.7°, 19.1°, 20.0°, 21.5°, 21.9°, and 23.4°±0.2° in 2θ.

In some embodiments, the mesylate salt of Form H is characterized by an X-ray powder diffraction pattern comprising at least three, at least four, at least five, at least six, at least seven, at least eight, or at least nine peaks at 8.1°, 10.1°, 11.7°, 13.7°, 19.1°, 20.0°, 20.8°, 21.5°, 21.9°, and 23.4°±0.2° in 2θ.

In some embodiments, the mesylate salt of Form H is characterized by an X-ray powder diffraction pattern comprising peaks at 8.1°, 10.1°, 11.7°, 13.7°, 19.1°, 20.0°, 20.8°, 21.5°, 21.9°, and 23.4°±0.2° in 2θ.

In some embodiments, the mesylate salt of Form H is characterized by an X-ray powder diffraction pattern comprising at least three, at least four, at least five, at least six, at least seven, at least eight, at least nine, at least ten, at least eleven, or at least twelve peaks at 8.1°, 10.1°, 10.7°, 11.7°, 13.7°, 14.6°, 19.1°, 20.0°, 20.8°, 21.5°, 21.9°, 23.4°, and 24.7°±0.2° in 2θ.

In some embodiments, the mesylate salt of Form H is characterized by an X-ray powder diffraction pattern comprising peaks at 8.1°, 10.1°, 10.7°, 11.7°, 13.7°, 14.6°, 19.1°, 20.0°, 20.8°, 21.5°, 21.9°, 23.4°, and 24.7°±0.2° in 2θ.

In some embodiments, the mesylate salt of Form H is characterized by an X-ray powder diffraction pattern comprising at least three or at least four peaks selected from 19.1°, 20.0°, 21.5°, 21.9° and 23.4° in 2θ. In some embodiments, the mesylate salt of Form H is characterized by an X-ray powder diffraction pattern comprising peaks at 19.1°, 20.0°, 21.5°, 21.9° and 23.4° in 2θ.

In some embodiments, the mesylate salt of Form H is characterized by an X-ray powder diffraction pattern comprising at least three or at least four peaks selected from 19.1°, 20.0°, 21.5°, 21.9° and 23.4°±0.2° in 2θ.

In some embodiments, the mesylate salt of Form H is characterized by an X-ray powder diffraction pattern comprising peaks at 19.1°, 20.0°, 21.5°, 21.9° and 23.4°±0.2° in 2θ.

In some embodiments, the mesylate salt of Form H is characterized by an X-ray powder diffraction pattern comprising at least three, at least four, at least five, at least six, at least seven, at least eight, or at least nine peaks at 11.7°, 13.6°, 14.6°, 19.1°, 20.0°, 20.8°, 21.5°, 21.9°, 23.4°, 24.6°±0.2° in 2θ.

In some embodiments, the mesylate salt of Form H is characterized by an X-ray powder diffraction pattern comprising peaks at 11.7°, 13.6°, 14.6°, 19.1°, 20.0°, 20.8°, 21.5°, 21.9°, 23.4°, 24.6°±0.2° in 2θ.

In some embodiments, the mesylate salt of Form H is characterized by an X-ray powder diffraction pattern comprising peaks at 7.0°, 8.1°, 10.1°, 10.7°, 11.7°, 12.3°, 12.7°, 13.2°, 13.7°, 13.9°, 14.6°, 14.9°, 16.2°, 16.8°, 17.1°, 17.9°, 18.1°, 18.8°, 19.1°, 19.2°, 20.0°, 20.6°, 20.8°, 20.9°, 21.0°, 21.5°, 21.9°, 22.1°, 22.7°, 22.9°, 23.2°, 23.4°, 23.5°, 24.0°, 24.7°, 24.7°, 25.6°, 26.5°, 26.8°, 27.8°, 28.2°, 29.0°, 29.7°, 30.0°, 32.1°, 32.6°, 34.0°, 37.3°, and 38.80°±0.2° in 2θ.

In some embodiments, the mesylate salt of Form H is characterized by an X-ray powder diffraction pattern comprising peaks at 6.9°, 8.1°, 10.1°, 10.7°, 11.7°, 12.3°, 12.7°, 13.2°, 13.6°, 13.9°, 14.6°, 14.9°, 16.2°, 16.8°, 17.1°, 17.9°, 18.1°, 18.8°, 19.1°, 19.7°, 20.0°, 20.6°, 20.8°, 21.0°, 21.5°, 21.9°, 22.1°, 22.8°, 23.2°, 23.4°, 24.1°, 24.3°, 24.6°, 25.4°, 25.6°, 26.4°, 26.8°, 27.1°, 27.8°, 28.2°, 29.0°, 29.6°, 30.0°, 31.2°, 31.8°, 32.1°, 32.6°, 33.3°, 34.0°, 34.3°, 34.8°, 35.6°, 37.3°, and 38.70°±0.2° in 2θ.

In some embodiments, the mesylate salt of Form H is characterized by an X-ray powder diffraction pattern substantially similar to FIG. 3A.

In some embodiments, the mesylate salt of Form H is characterized by differential scanning calorimeter (DSC) thermogram comprising an endotherm onset at 220.5° C.±2° C., and an exotherm onset at 239.4° C.±2° C. In some embodiments, the mesylate salt of Form H is characterized by differential scanning calorimeter (DSC) thermogram substantially similar to that in FIG. 3B.

In some embodiments, the mesylate salt of Form H is characterized by a thermogravimetric analysis (TGA) substantially similar to that in FIG. 3B.

In some embodiments, the mesylate salt of Form H is characterized by a differential scanning calorimeter (DSC) thermogram comprising an endotherm onset at 228.7° C.±2° C. In some embodiments, the DSC was performed using a hermetic aluminum pan with pinhole.

### Mesylate Salt of Form I

In some embodiments, the present disclosure provides a crystalline Form I of mesylate salt of Compound (I), wherein the molar ratio between Compound (I) and methanesulfonic acid is 1:1.

In some embodiments, crystalline Form I of mesylate salt of Compound (I) is a hydrate.

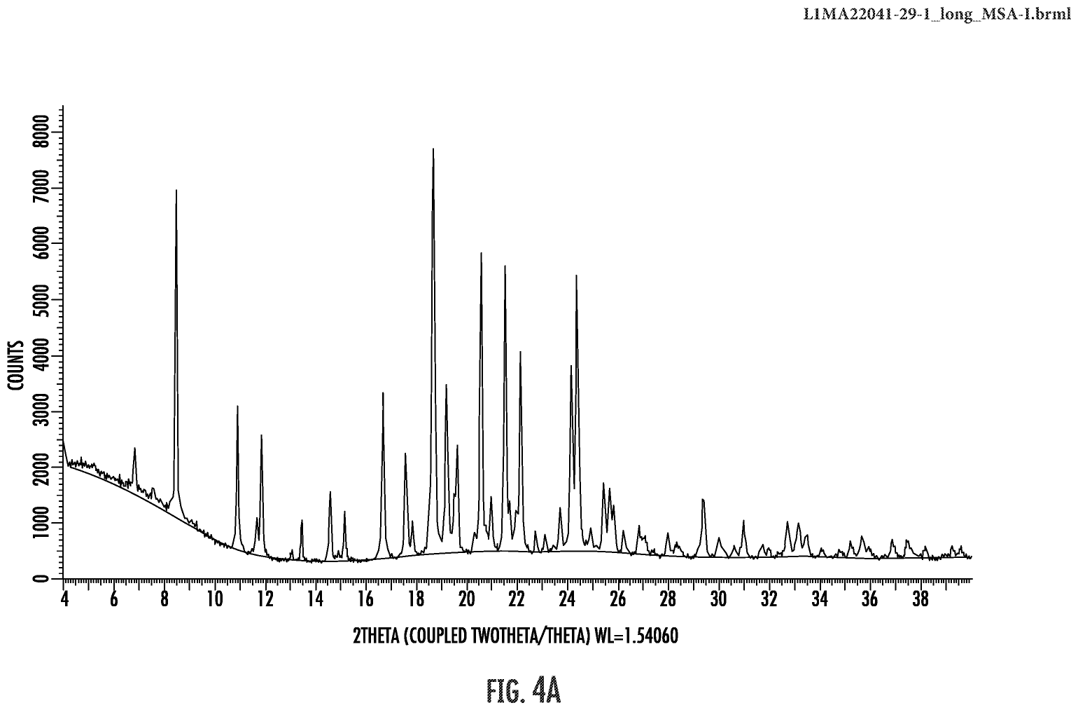

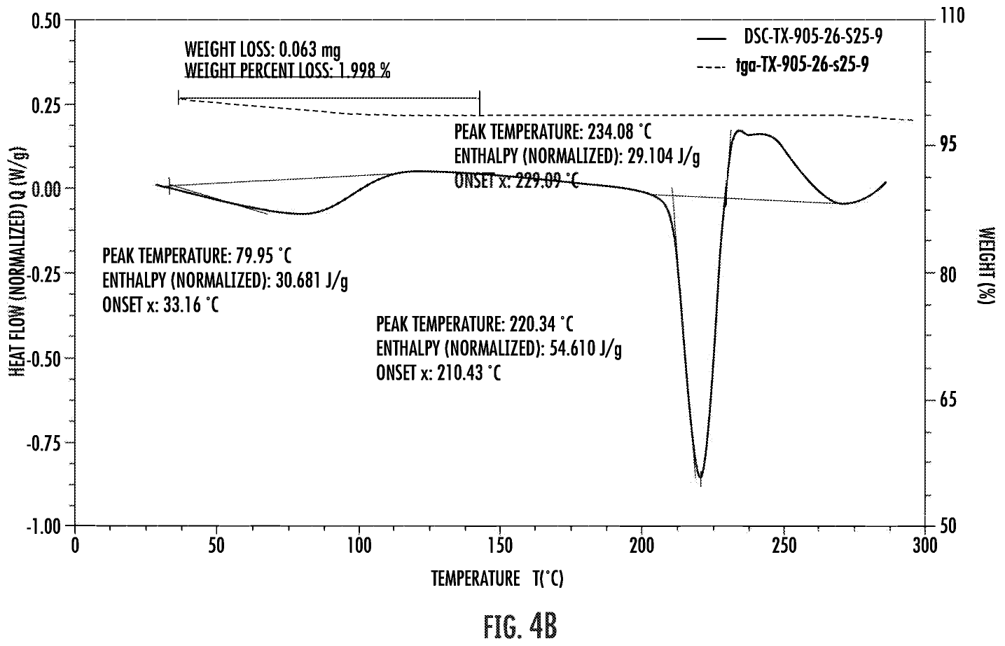

The XRPD pattern and peaks are shown in FIG. 4A, and the Thermogravimetric Analysis (TGA) and Differential Scanning Calorimetry Analysis (DSC) thermograms are shown in FIG. 4B.

TABLE 11a
Peak list for Compound (I) mesylate Form I

| 2-theta | d-Spacing | Relative |
|---|---|---|
| (deg | (Å) | Intensity |
| 8.57 | 10.31 | 44 |
| 10.98 | 8.05 | 48 |
| 11.92 | 7.42 | 36 |
| 16.71 | 5.30 | 44 |
| 18.72 | 4.74 | 100 |
| 19.27 | 4.60 | 45 |
| 20.61 | 4.31 | 74 |
| 21.58 | 4.11 | 42 |
| 22.17 | 4.01 | 77 |
| 24.17 | 3.68 | 41 |
| 24.41 | 3.64 | 58 |

TABLE 11b
Peak list for Compound (I) mesylate Form I

| 2-theta | d-spacing | Relative |
|---|---|---|
| (deg.) | (ang.) | intensity |
| 6.82 | 12.94 | 11 |
| 8.49 | 10.40 | 81 |
| 10.91 | 8.10 | 35 |
| 11.66 | 7.58 | 9 |
| 11.87 | 7.45 | 29 |
| 13.45 | 6.58 | 10 |
| 14.59 | 6.07 | 17 |
| 15.16 | 5.84 | 12 |
| 16.68 | 5.31 | 41 |
| 17.58 | 5.04 | 25 |
| 17.85 | 4.96 | 9 |
| 18.67 | 4.75 | 100 |
| 19.19 | 4.62 | 41 |
| 19.51 | 4.55 | 15 |
| 19.62 | 4.52 | 27 |
| 20.31 | 4.37 | 5 |
| 20.56 | 4.32 | 73 |
| 20.96 | 4.24 | 14 |
| 21.51 | 4.13 | 70 |
| 21.69 | 4.09 | 12 |
| 21.97 | 4.04 | 10 |
| 22.13 | 4.01 | 49 |
| 22.73 | 3.91 | 5 |
| 23.71 | 3.75 | 10 |
| 24.14 | 3.68 | 45 |
| 24.36 | 3.65 | 67 |
| 24.90 | 3.57 | 5 |
| 25.42 | 3.50 | 17 |
| 25.66 | 3.47 | 15 |
| 25.81 | 3.45 | 11 |
| 26.21 | 3.40 | 5 |
| 26.83 | 3.32 | 7 |
| 27.97 | 3.19 | 6 |
| 29.38 | 3.04 | 14 |
| 29.98 | 2.98 | 5 |
| 30.97 | 2.88 | 9 |
| 32.71 | 2.74 | 8 |
| 33.14 | 2.70 | 8 |
| 33.48 | 2.67 | 5 |
| 35.66 | 2.52 | 5 |

In Tables 11a and 11b only those peaks with a relative intensity of five or greater compared to the absolute intensity (I. in cps°) of the most intense peak are reported. Tables 11a and 11b XRPD peaks obtained from two different batches of crystalline Form I of mesylate salt of Compound (I). Table 12a are condensed peak lists selected from those in Table 11a and Tables 12b and 12c are condensed peak lists selected from those in Table 11b.

TABLE 12a
Condensed peak list #1 for Compound (I) mesylate Form I

| 2-theta | d-Spacing | Relative |
|---|---|---|
| (deg) | (Å) | Intensity |
| 8.57 | 10.31 | 44 |
| 10.98 | 8.05 | 48 |
| 16.71 | 5.30 | 44 |
| 18.72 | 4.74 | 100 |
| 19.27 | 4.60 | 45 |
| 20.61 | 4.31 | 74 |
| 21.58 | 4.11 | 42 |
| 22.17 | 4.01 | 77 |
| 24.17 | 3.68 | 41 |
| 24.41 | 3.64 | 58 |

TABLE 12b
Condensed peak list #1 for Compound (I) mesylate Form I

| | d-spacing | Relative |
|---|---|---|
| 2-theta (deg.) | (ang.) | intensity |
| 18.67 | 4.75 | 100 |
| 8.49 | 10.40 | 81 |
| 20.56 | 4.32 | 73 |
| 21.51 | 4.13 | 70 |
| 24.36 | 3.65 | 67 |
| 22.13 | 4.01 | 49 |
| 24.14 | 3.68 | 45 |
| 16.68 | 5.31 | 41 |
| 19.19 | 4.62 | 41 |
| 10.91 | 8.10 | 35 |

TABLE 12c
Condensed peak list #2 for Compound (I) mesylate Form I

| 2-theta | d-spacing | Relative |
|---|---|---|
| (deg.) | (ang.) | intensity |
| 18.67 | 4.75 | 100 |
| 8.49 | 10.40 | 81 |
| 20.56 | 4.32 | 73 |
| 21.51 | 4.13 | 70 |
| 24.36 | 3.65 | 67 |

In some embodiments, the mesylate salt of Form I is characterized by an X-ray powder diffraction pattern comprising at least three or at least four peaks selected from 11.0°, 18.7°, 20.6°, 22.2°, and 24.4°±0.2° in 2θ.

In some embodiments, the mesylate salt of Form I is characterized by an X-ray powder diffraction pattern comprising peaks at 11.0°, 18.7°, 20.6°, 22.2°, and 24.4°±0.2° in 2θ.

In some embodiments, the mesylate salt of Form I is characterized by an X-ray powder diffraction pattern comprising at least three, at least four, at least five, at least six, at least seven, at least eight, or at least nine peaks at 8.6°, 11.0°, 16.7°, 18.7°, 19.3°, 20.6°, 21.6°, 22.2°, 24.2°, and 24.4°±0.2° in 2θ.

In some embodiments, the mesylate salt of Form I is characterized by an X-ray powder diffraction pattern comprising peaks at 8.6°, 11.0°, 16.7°, 18.7°, 19.3°, 20.6°, 21.6°, 22.2°, 24.2°, and 24.4°±0.2° in 2θ.

In some embodiments, the mesylate salt of Form I is characterized by an X-ray powder diffraction pattern comprising at least three, at least four, at least five, at least six, at least seven, at least eight, at least nine, or at least ten peaks at 8.6°, 11.0°, 11.9°, 16.7°, 18.7°, 19.3°, 20.6°, 21.6°, 22.2°, 24.2°, and 24.4°±0.2° in 2θ.

In some embodiments, the mesylate salt of Form I is characterized by an X-ray powder diffraction pattern comprising peaks at 8.6°, 11.0°, 11.9°, 16.7°, 18.7°, 19.3°, 20.6°, 21.6°, 22.2°, 24.2°, and 24.4°±0.2° in 2θ.

In some embodiments, the mesylate salt of Form I is characterized by an X-ray powder diffraction pattern comprising at least three or at least four peaks selected from 8.5°, 18.7°, 20.6°, 21.5°, and 24.4°±0.2° in 2θ.

In some embodiments, the mesylate salt of Form I is characterized by an X-ray powder diffraction pattern comprising peaks at 8.5°, 18.7°, 20.6°, 21.5°, and 24.4°±0.2° in 2θ.

In some embodiments, the mesylate salt of Form I is characterized by an X-ray powder diffraction pattern comprising at least three, at least four, at least five, at least six, at least seven, at least eight, or at least nine peaks at 8.5°, 10.9°, 16.7°, 18.7°, 19.2°, 20.6°, 21.5°, 22.1°, 24.1°, and 24.4°±0.2° in 2θ.

In some embodiments, the mesylate salt of Form I is characterized by an X-ray powder diffraction pattern comprising peaks at 8.5°, 10.9°, 16.7°, 18.7°, 19.2°, 20.6°, 21.5°, 22.1°, 24.10, and 24.4°±0.2° in 2θ.

In some embodiments, the mesylate salt of Form I is characterized by an X-ray powder diffraction pattern comprising peaks at 6.8°, 8.5°, 10.9°, 11.7°, 11.9°, 13.5°, 14.6°, 15.2°, 16.7°, 17.6°, 17.9°, 18.7°, 19.2°, 19.5°, 19.6°, 20.3°, 20.6°, 21.0°, 21.5°, 21.7°, 22.0°, 22.1°, 22.7°, 23.7°, 24.1°, 24.4°, 24.9°, 25.4°, 25.7°, 25.8°, 26.2°, 26.8°, 28.0°, 29.4°, 30.0°, 31.0°, 32.7°, 33.1°, 33.5° and 35.7°±0.2° in 2θ.

In some embodiments, the mesylate salt of Form I is characterized by an X-ray powder diffraction pattern substantially similar to FIG. 4A.

In some embodiments, the mesylate salt of Form I is characterized by differential scanning calorimeter (DSC) thermogram comprising an endotherm onset at 33.2° C.±2° C., an endotherm onset at 210.4° C.±2° C., and an exotherm onset at 229.1° C.±2° C. In some embodiments, the mesylate salt of Form I is characterized by a differential scanning calorimeter (DSC) thermogram substantially similar to that in FIG. 4B.

In some embodiments, the mesylate salt of Form I is characterized by a thermogravimetric analysis (TGA) substantially similar to that in FIG. 4B.

In some embodiments, the mesylate salt of Form I is characterized by a differential scanning calorimeter (DSC) thermogram comprising an endotherm onset at 221.3° C.±2° C. In some embodiments, the DSC thermogram further comprises an endotherm onset at 34.3° C.±2° C. In some embodiments, the DSC was performed using a hermetic aluminum pan with pinhole.

Free Bases of Compound (I) In one aspect, the present disclosure provides free base of Compound (I).

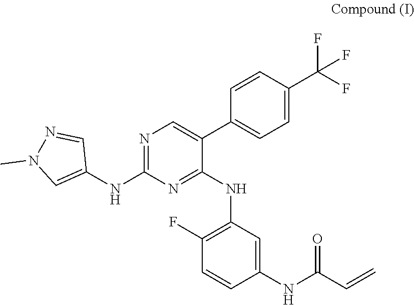 [SMILES: C=CC(=O)Nc1ccc(F)c(Nc2nc(Nc3cnn(C)c3)ncc2-c2ccc(C(F)(F)F)cc2)c1]

In some embodiments, the free base of Compound (I) is in an amorphous form.

In some embodiments, the free base of Compound (I) is crystalline. In some embodiments, the free base of Compound (I) is in a single crystalline form.

In some embodiments, the free base of Compound (I) is unsolvated. In other embodiments, the free base of Compound (I) is solvated.

### Free Base Crystalline Form A

In some embodiments, the present disclosure provides a free base crystalline Form A of Compound (I). In some embodiments, crystalline Form A of Compound (I) free base is an anhydrate.

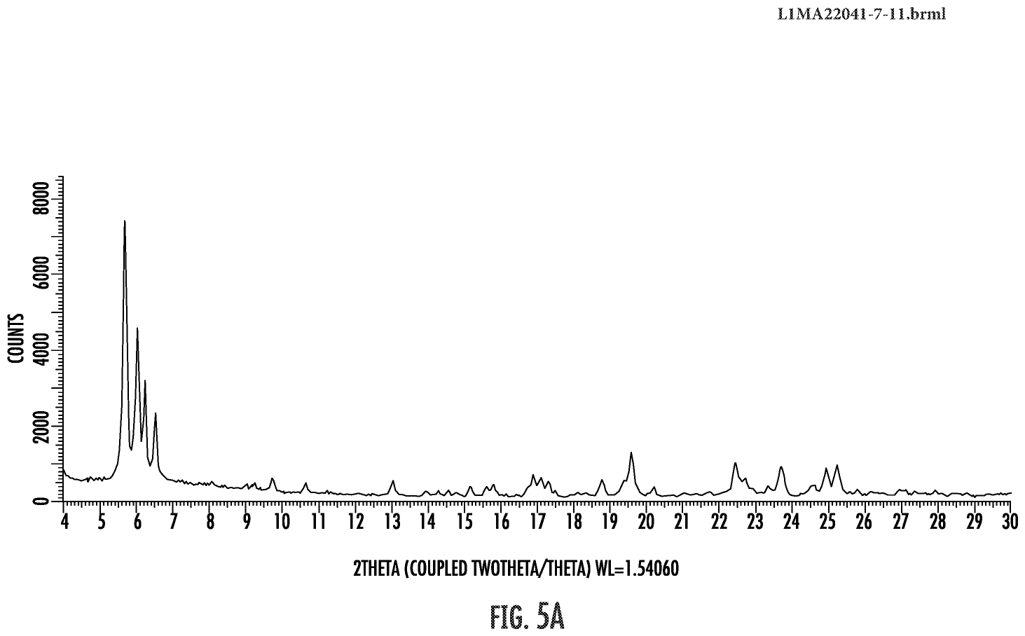

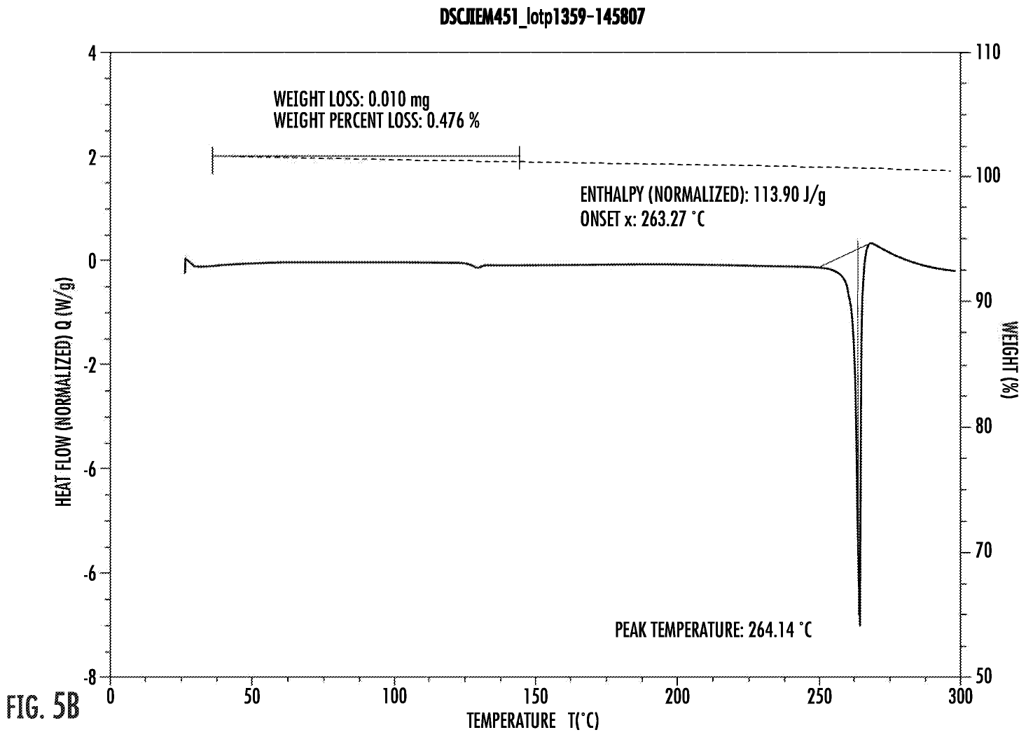

The XRPD pattern and peaks are shown in FIG. 5A, and the Thermogravimetric Analysis (TGA) and Differential Scanning Calorimetry Analysis (DSC) thermograms are shown in FIG. 5B.

TABLE 13a
Peak list for Compound (I) free base Form A

| 2-theta | d-Spacing | Relative |
|---|---|---|
| (deg) | (Å) | Intensity |
| 5.66 | 5.66 | 100 |
| 5.99 | 5.99 | 14 |
| 6.20 | 6.20 | 11 |

TABLE 13b
- Peak list for Compound (I) free base Form A

| 2-theta | d-spacing | Relative |
|---|---|---|
| (deg.) | (ang.) | intensity |
| 5.70 | 15.50 | 100 |
| 6.04 | 14.63 | 58 |
| 6.23 | 14.17 | 39 |
| 6.52 | 13.55 | 27 |
| 9.73 | 9.08 | 5 |
| 13.02 | 6.79 | 5 |
| 15.79 | 5.61 | 5 |
| 16.89 | 5.25 | 8 |
| 17.08 | 5.19 | 7 |
| 17.30 | 5.12 | 6 |
| 18.78 | 4.72 | 6 |
| 19.45 | 4.56 | 6 |
| 19.58 | 4.53 | 17 |
| 22.44 | 3.96 | 13 |
| 22.69 | 3.92 | 6 |
| 23.70 | 3.75 | 11 |
| 24.93 | 3.57 | 10 |
| 25.23 | 3.53 | 11 |

In Tables 13a and 13b only those peaks with a relative intensity of five or greater compared to the absolute intensity (I. in cps°) of the most intense peak are reported. Tables 13a and 13b XRPD peaks obtained from two different batches of crystalline Form A of Compound (I) free base. Tables 13c and 13d are condensed peak lists selected from those in Table 13b.

TABLE 13c
Condensed peak list #1 for Compound (I) free base Form A

| 2-theta | d-spacing | Relative |
|---|---|---|
| (deg.) | (ang.) | intensity |
| 5.70 | 15.50 | 100 |
| 6.04 | 14.63 | 58 |
| 6.23 | 14.17 | 39 |
| 6.52 | 13.55 | 27 |
| 19.58 | 4.53 | 17 |
| 22.44 | 3.96 | 13 |
| 23.70 | 3.75 | 11 |
| 25.23 | 3.53 | 11 |
| 24.93 | 3.57 | 10 |
| 16.89 | 5.25 | 8 |

TABLE 13d
Condensed peak list #2 for Compound (I) free base Form A

| 2-theta | d-spacing | Relative |
|---|---|---|
| (deg.) | (ang.) | intensity |
| 5.70 | 15.50 | 100 |
| 6.04 | 14.63 | 58 |
| 6.23 | 14.17 | 39 |
| 6.52 | 13.55 | 27 |
| 19.58 | 4.53 | 17 |

In some embodiments, the free base crystalline Form A is characterized by an X-ray powder diffraction pattern comprising peaks at 5.7°, 6.0° and 6.20°±0.2 in 2θ.

In some embodiments, the free base crystalline Form A is characterized by an X-ray powder diffraction pattern comprising at least three or at least four peaks selected from 5.7°, 6.0°, 6.2°, 6.5° and 19.60°±0.2° in 2θ.

In some embodiments, the free base crystalline Form A is characterized by an X-ray powder diffraction pattern comprising peaks at 5.7°, 6.0°, 6.2°, 6.5° and 19.6°±0.2 in 2θ.

In some embodiments, the free base crystalline Form A is characterized by an X-ray powder diffraction pattern comprising at least three, at least four, at least five, at least six, at least seven, at least eight, or at least nine peaks at 5.7°, 6.0°, 6.2°, 6.5°, 16.9°, 19.6°, 22.4°, 23.7°, 24.9° and 25.2°±0.2° in 2θ.

In some embodiments, the free base crystalline Form A is characterized by an X-ray powder diffraction pattern comprising peaks at 5.7°, 6.0°, 6.2°, 6.5°, 16.9°, 19.6°, 22.4°, 23.7°, 24.9° and 25.2°±0.2° in 2θ.

In some embodiments, the free base crystalline Form A is characterized by an X-ray powder diffraction pattern comprising peaks at 5.7°, 6.0°, 6.2°, 6.5°, 9.7°, 13.0°, 15.8°, 16.9°, 17.1°, 17.3°, 18.8°, 19.5°, 19.6°, 22.4°, 22.7°, 23.7°, 24.9° and 25.2°±0.2° in 2θ.

In some embodiments, the free base crystalline Form A is characterized by an X-ray powder diffraction pattern substantially similar to FIG. 5A.

In some embodiments, the free base crystalline Form A is characterized by differential scanning calorimeter (DSC) thermogram comprising an endotherm onset at 263.3° C.±2° C. In some embodiments, the free base crystalline Form A is characterized by a differential scanning calorimeter (DSC) thermogram substantially similar to that in FIG. 5B. In some embodiments, the DSC thermogram further comprises an endotherm onset at 156.5° C.±2° C. In some embodiments, the DSC were performed using a hermetic aluminum pan with pinhole.

In some embodiments, the free base crystalline Form A s characterized by a thermogravimetric analysis (TGA) substantially similar to that in FIG. 5B.

### Free Base Crystalline Form B

In some embodiments, the present disclosure provides a free base crystalline Form B of Compound (I). In some embodiments, crystalline Form B of Compound (I) free base is a hydrate.

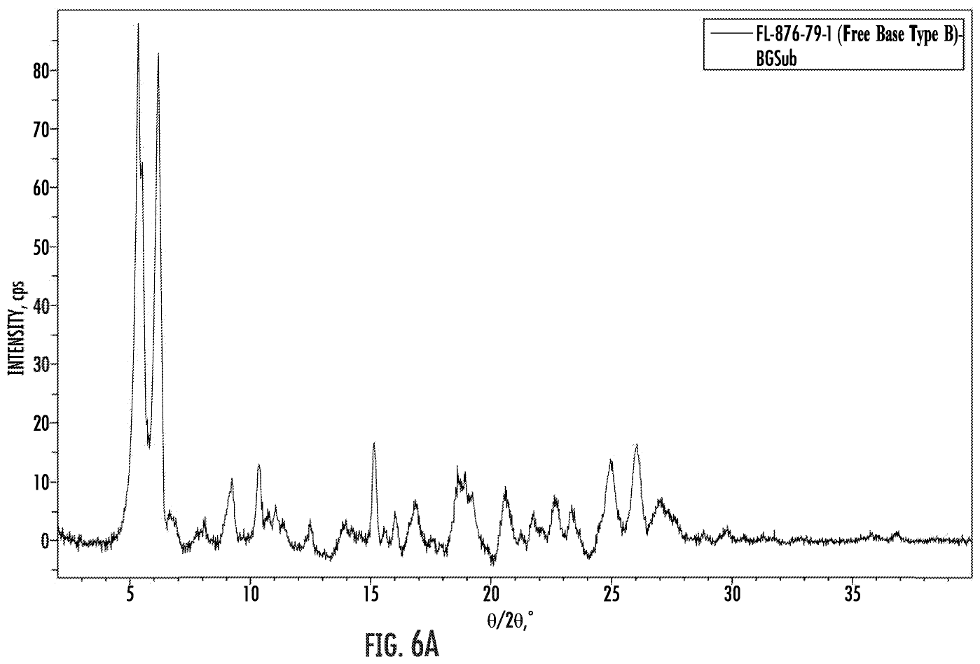

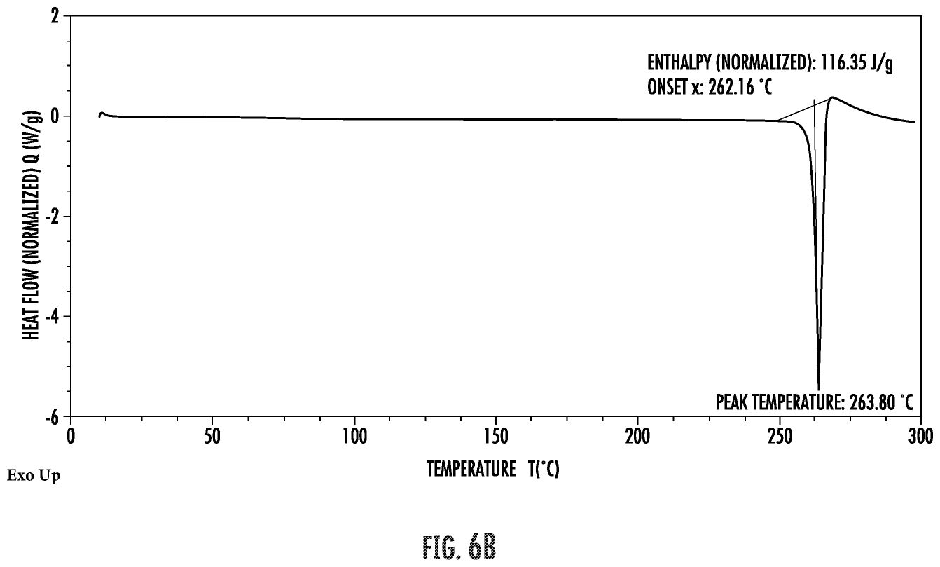

The XRPD pattern and peaks are shown in FIG. 6A.

TABLE 14a
Peak list for Compound (I) free base Form B

| 2-theta | d-Spacing | Relative |
|---|---|---|
| (deg) | (Å) | Intensity |
| 5.38 | 16.40 | 100 |
| 5.70 | 15.49 | 78 |
| 6.17 | 14.31 | 78 |
| 9.22 | 9.58 | 15 |
| 10.34 | 8.54 | 16 |
| 15.14 | 5.85 | 15 |
| 18.60 | 4.77 | 16 |
| 19.07 | 4.65 | 29 |
| 20.61 | 4.31 | 17 |
| 24.95 | 3.57 | 37 |
| 26.02 | 3.42 | 31 |
| 27.11 | 3.29 | 24 |

*peak at 5.7 was included by machine error.

TABLE 14b
Peak list for Compound (I) free base Form B

| 2-theta | d-spacing | Relative |
|---|---|---|
| (deg.) | (ang.) | intensity |
| 5.16 | 17.1 | 100 |
| 5.34 | 16.5 | 64 |
| 6.13 | 14.4 | 92 |
| 8.11 | 10.9 | 6 |
| 9.12 | 9.7 | 8 |
| 13.78 | 6.4 | 5 |
| 14.22 | 6.2 | 9 |
| 15.58 | 5.7 | 12 |
| 16.56 | 5.3 | 5 |
| 17.30 | 5.1 | 5 |
| 18.46 | 4.8 | 13 |
| 18.68 | 4.7 | 12 |
| 19.53 | 4.5 | 11 |
| 21.87 | 4.1 | 6 |
| 22.53 | 3.9 | 10 |
| 23.11 | 3.8 | 6 |
| 24.41 | 3.6 | 21 |
| 24.89 | 3.6 | 10 |
| 25.37 | 3.5 | 7 |
| 26.08 | 3.4 | 11 |
| 26.18 | 3.4 | 9 |
| 26.64 | 3.3 | 6 |
| 27.10 | 3.3 | 7 |

In Tables 14a and 14b only those peaks with a relative intensity of five or greater compared to the absolute intensity (I. in cps°) of the most intense peak are reported. Tables 14a and 14b are XRPD peaks obtained from two different batches of free base Form B of Compound (I). Tables 15a and 16a are condensed peak lists selected from those in Table 14a and Tables 15b and 16b are condensed peak lists selected from those in Table 14b.

TABLE 15a
Condensed peak list #1 for Compound (I) free base Form B

| 2-theta | d-Spacing | Relative |
|---|---|---|
| (deg) | (Å) | Intensity |
| 5.38 | 16.40 | 100 |
| *5.70 | 15.49 | 78 |
| 6.17 | 14.31 | 78 |
| 19.07 | 4.65 | 29 |
| 24.95 | 3.57 | 37 |
| 26.02 | 3.42 | 31 |
| 27.11 | 3.29 | 24 |

*peak at 5.7 was included by machine error.

TABLE 15b
Condensed peak list #1 for Compound (I) free base Form B

| 2-theta | d-spacing | Relative |
|---|---|---|
| (deg.) | (ang.) | intensity |
| 5.16 | 17.1 | 100 |
| 6.13 | 14.4 | 92 |
| 5.34 | 16.5 | 64 |
| 24.41 | 3.6 | 21 |
| 18.46 | 4.8 | 13 |
| 15.58 | 5.7 | 12 |
| 18.68 | 4.7 | 12 |
| 19.53 | 4.5 | 11 |
| 26.08 | 3.4 | 11 |
| 22.53 | 3.9 | 10 |

TABLE 16a
Condensed peak list #2 for Compound (I) free base Form B

| 2-theta | d-Spacing | Relative |
|---|---|---|
| (deg) | (Å) | Intensity |
| 5.38 | 16.40 | 100 |
| *5.70 | 15.49 | 78 |
| 6.17 | 14.31 | 78 |
| 24.95 | 3.57 | 37 |
| 26.02 | 3.42 | 31 |

*peak at 5.7 was included by machine error.

TABLE 16b
Condensed peak list #2 for Compound (I) free base Form B

| 2-theta | d-spacing | Relative |
|---|---|---|
| (deg.) | (ang.) | intensity |
| 5.16 | 17.1 | 100 |
| 6.13 | 14.4 | 92 |
| 5.34 | 16.5 | 64 |
| 24.41 | 3.6 | 21 |
| 18.46 | 4.8 | 13 |

In some embodiments, the free base crystalline Form B is characterized by an X-ray powder diffraction pattern comprising at least three or at least four peaks selected from 5.2°, 5.3°, 6.1°, 18.5° and 24.4°±0.2° in 2θ.

In some embodiments, the free base crystalline Form B is characterized by an X-ray powder diffraction pattern comprising peaks at 5.2°, 5.3°, 6.1°, 18.5° and 24.4°±0.2° in 2θ.

In some embodiments, the free base crystalline Form B is characterized by an X-ray powder diffraction pattern comprising at least three, at least four, at least five, at least six, at least seven, at least eight, or at least nine peaks at 5.2°, 5.3°, 6.1°, 15.6°, 18.5°, 18.7°, 19.5°, 22.5°, 24.4° and 26.1°±0.2° in 2θ.

In some embodiments, the free base crystalline Form B is characterized by an X-ray powder diffraction pattern comprising peaks at 5.2°, 5.3°, 6.1°, 15.6°, 18.5°, 18.7°, 19.5°, 22.5°, 24.4° and 26.1°±0.2° in 2θ.

In some embodiments, the free base crystalline Form B is characterized by an X-ray powder diffraction pattern comprising peaks at 5.4°, 6.2°, 9.2°, 10.3°, 15.1°, 18.6°, 19.1°, 20.6°, 25.0°, 26.0°, and 27.1°±0.2° in 2θ.

In some embodiments, the free base crystalline Form B is characterized by an X-ray powder diffraction pattern comprising peaks at 5.2°, 5.3°, 6.1°, 8.1°, 9.1°, 13.8°, 14.2°, 15.6°, 16.6°, 17.3°, 18.5°, 18.7°, 19.5°, 21.9°, 22.5°, 23.1°, 24.4°, 24.9°, 25.4°, 26.1°, 26.2°, 26.6° and 27.1°±0.2° in 2θ.

In some embodiments, the free base crystalline Form B is characterized by an X-ray powder diffraction pattern substantially similar to FIG. 6A.

In some embodiments, the free base crystalline Form B is characterized by an characterized by differential scanning calorimeter (DSC) thermogram comprising an endotherm onset at 262.2° C.±2° C. In some embodiments, the free base crystalline Form A is characterized by a differential scanning calorimeter (DSC) thermogram substantially similar to that in FIG. 6B. In some embodiments, the DSC was performed using a hermetic aluminum pan with pinhole.

### Pharmaceutical Compositions

Pharmaceutical compositions of the disclosure (also referred to herein as the “disclosed pharmaceutical compositions”) comprise a pharmaceutically acceptable carrier and a salt or solid form of the disclosure.

Some embodiments of the disclosure relate to a pharmaceutical composition comprising: a pharmaceutically acceptable carrier; and a mesylate salt of Compound (I), wherein the molar ratio between Compound (I) and methanesulfonic acid is about 1:1. In some embodiments, the mesylate salt is crystalline. In some embodiments, the mesylate salt of Compound (I) is crystalline Form A.

Some embodiments of the disclosure relate to a pharmaceutical composition comprising: a pharmaceutically acceptable carrier; and a mesylate salt of Compound (I), wherein the molar ratio between Compound (I) and methanesulfonic acid is about 1:1. In some embodiments, the mesylate salt is crystalline. In some embodiments, the mesylate salt of Compound (I) is crystalline Form B.

Some embodiments of the disclosure relate to a pharmaceutical composition comprising: a pharmaceutically acceptable carrier; and a mesylate salt of Compound (I). In some embodiments, the mesylate salt is crystalline. In some embodiments, the mesylate salt of Compound (I) is crystalline Form H.

Some embodiments of the disclosure relate to a pharmaceutical composition comprising: a pharmaceutically acceptable carrier; and a mesylate salt of Compound (I). In some embodiments, the mesylate salt is crystalline. In some embodiments, the mesylate salt of Compound (I) is crystalline Form I.

Some embodiments of the disclosure relate to a pharmaceutical composition comprising: a pharmaceutically acceptable carrier; and Compound (I) free base. In some embodiments, the free base is crystalline. In some embodiments, the free base of Compound (I) is crystalline Form A. In some embodiments, the free base of Compound (I) is crystalline Form B.

Salts or solid forms of the disclosure may be formulated for administration in any convenient way for use in human or veterinary medicine. In some embodiments, the compound or salt included in the pharmaceutical compositions may be active itself, or may be a prodrug, e.g., capable of being converted to an active compound in a physiological setting.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutical composition” refers to one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition comprising a compound of the present disclosure and a pharmaceutically acceptable carrier.

“Carrier” refers to a diluent, adjuvant, excipient, or vehicle with which the active ingredient is administered. In some embodiments, such pharmaceutical carriers are sterile liquids, such as water and oils, including those of petroleum, animal, vegetable or synthetic origin, including but not limited to peanut oil, soybean oil, mineral oil, sesame oil and the like. In some embodiments, water is a carrier when the pharmaceutical composition is administered orally. In some embodiments, saline and aqueous dextrose are exemplary carriers when the pharmaceutical composition is administered intravenously. In some embodiments, saline solutions and aqueous dextrose and glycerol solutions are employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In some embodiments, the pharmaceutical composition comprises minor amounts of wetting or emulsifying agents, or pH buffering agents. In some embodiments, these pharmaceutical compositions take the form of solutions, suspensions, emulsions, tablets, pills, capsules, powders, sustained-release formulations and the like. In some embodiments, the pharmaceutical composition is formulated as a suppository, with traditional binders and carriers such as triglycerides. In some embodiments, an oral formulation comprises carriers such as pharmaceutical grades of mannitol, lactose, starch, magnesium stearate, sodium saccharine, cellulose, magnesium carbonate, etc. Examples of suitable pharmaceutically acceptable carriers are described in “Remington's Pharmaceutical Sciences” by E. W. Martin. Such pharmaceutical compositions will contain a therapeutically effective amount of the active ingredient, for example in purified form, together with a suitable amount of carrier so as to provide the form for proper administration to the subject. The formulation should suit the mode of administration.

### Methods of Treatment

Some embodiments provided herein describe different solid forms and salt forms of Compound (I) that are useful as epidermal growth factor receptor (EGFR) family kinase inhibitors. In some embodiments, solid forms and salt forms of Compound (I) are useful as mutant EGFR family kinase inhibitors. In some embodiments, the

In some embodiments, the solid forms and salt forms of Compound (I) described herein have improved safety profiles. In some embodiments, the solid forms and salt forms of Compound (I) described herein have improved toxicity profile. In some embodiments, the solid forms and salt forms of Compound (I) described herein have an improved therapeutic index. In some embodiments, the solid forms and salt forms of Compound (I) described herein have improved antitumor activity against brain metastasis.

In some embodiments, the presence of the EGFR family kinase mutants is determined by the assessment of archival tumor biopsy or blood sample. In some embodiments, EGFR family kinase mutants are detected with a commercially available test kit. In some embodiments, EGFR family kinase mutants are detected with a reverse transcription polymerase chain reaction (RT-PCR)-based method. In some embodiments, EGFR family kinase mutants are detected with a sequencing-based method. In some embodiments, EGFR family kinase mutants are detected with a mass spectrometry genotyping-based method. In some embodiments, EGFR family kinase mutants are detected with an immunohistochemistry-based method. In some embodiments, EGFR family kinase mutants are detected with a molecular diagnostics panel. In some embodiments, EGFR family kinase mutants are detected from a tumor sample. In some embodiments, EGFR family kinase mutants are detected from circulating DNA. In some embodiments, EGFR family kinase mutants are detected from tumor cells.

In one aspect, provided herein is a method of inhibiting an EGFR family kinase mutant in a subject in need thereof, comprising administering to the subject a therapeutically effective amount of a solid form or a salt form of Compound (I).

In another aspect, provided herein is a method of inhibiting a drug-resistant EGFR mutant in a subject in need thereof, comprising administering to the subject a therapeutically effective amount of a solid form or a salt form of Compound (I). In some embodiments, the drug-resistant EGFR mutant is del19/T790M EGFR or L858R/T790M EGFR.

In another aspect, provided herein is a method of inhibiting EGFR in a subject in need thereof, comprising administering to the subject a therapeutically effective amount of a solid form or a salt form of Compound (I), wherein the compound exhibits greater inhibition of an EGFR mutant relative to wild-type EGFR.

In some embodiments, the EGFR mutant comprises a substitution in exon 18, a deletion in exon 19, a substitution in exon 20, an insertion in exon 20, a mutation in the extracellular domain, or a substitution in exon 21. In some embodiments, the EGFR mutant is selected from del19/T790M EGFR, L858R/T790M EGFR, L858R EGFR, L861Q EGFR, S768I EGFR, G719X EGFR, 763insFQEA EGFR, 767insTLA EGFR, 769insASV EGFR, 769insGE EGFR, 770insSVD EGFR (or D770_N771insSVD EGFR), 770insNPG EGFR (or D770_N771insNPG EGFR), 770insGT EGFR, 770insGF EGFR, 770insG EGFR, 771insH EGFR, 771insN EGFR, 772insNP EGFR, 773insNPH EGFR (or H773insNPH EGFR), 773insH EGFR, 773insPH EGFR, EGFRvii, EGFRviii, A767_dupASV EGFR, 773insAH EGFR, M766_A767insAI EGFR, and any combination thereof. In some embodiments, the EGFR mutant is del19/T790M EGFR or L858R/T790M EGFR. In some embodiments, the EGFR mutant is del19/T790M EGFR. In some embodiments, the EGFR mutant is L858R/T790M EGFR. In some embodiments, the EGFR mutant is an insertion in exon 20. In some embodiments, the EGFR mutation is an exon 18 G719X or exon 21 L861Q mutation. In some embodiments, the mutation is S768I EGFR.

In another aspect, provided herein is a method of treating a disease associated EGFR in a subject in need thereof, comprising administering to the subject a therapeutically effective amount of a solid form or a salt form of Compound (I).

In some embodiments, the disease in the subject comprises an EGFR mutation (i.e., the disease in the subject is characterized by an EGFR mutation). In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is selected from del19/T790M EGFR, L858R/T790M EGFR, L858R EGFR, L861Q EGFR, S768I EGFR, G719X EGFR, 763insFQEA EGFR, 767insTLA EGFR, 769insASV EGFR, 769insGE EGFR, 770insSVD EGFR (or D770_N771insSVD EGFR), 770insNPG EGFR (or D770_N771insNPG EGFR), 770insGT EGFR, 770insGF EGFR, 770insG EGFR, 771insH EGFR, 771insN EGFR, 772insNP EGFR, 773insNPH EGFR (or H773insNPH EGFR), 773insH EGFR, 773insPH EGFR, EGFRvii, EGFRviii, A767_dupASV EGFR, 773insAH EGFR, M766_A767insAI EGFR, and any combination thereof. In some embodiments, the EGFR mutation is del19/T790M EGFR or L858R/T790M EGFR. In some embodiments, the EGFR mutation is del19/T790M EGFR. In some embodiments, the EGFR mutation is L858R/T790M EGFR. In some embodiments, the EGFR mutant is an insertion in exon 20. In some embodiments, the EGFR mutant is an exon 18 G719X or exon 21 L861Q mutation. In some embodiments, the mutation is S768I EGFR.

In another aspect, provided herein is a method of treating cancer in a subject in need thereof, comprising administering to the subject a therapeutically effective amount of a solid form or a salt form of Compound (I). In some embodiments, the cancer is incurable recurrent cancer.

In some embodiments, the cancer is locally advanced or metastatic disease. In one aspect, the subject is an adult. In one aspect, the treating is first-line treatment. In one aspect, the treating is second-line treatment. In one aspect, the subject has previously been treated with platinum-based chemotherapy. In one aspect, the subject's disease has progressed on or after platinum-based chemotherapy. In one aspect, the subject has previously been treated with at least one systemic prior treatment. In one aspect, the subject has previously been treated with an EGFR exon 20 insertion targeted agent. In one aspect, the treating is adjuvant treatment after tumor resection. In some embodiments, the cancer displays drug resistance associated with EGFR del19/T790M activation. In some embodiments, the cancer displays drug resistance associated with EGFR L858R/T790M activation. In some embodiments, the cancer is characterized by an EGFR mutation. In some embodiments, the cancer is characterized by an insertion in exon 20. In some embodiments, the cancer is characterized by an exon 18 G719X or exon 21 L861Q mutation.

In some embodiments, the cancer is bladder cancer, prostate cancer, breast cancer, cervical cancer, colorectal cancer, endometrial cancer, gastric cancer, glioblastoma, head and neck cancer, lung cancer, urothelial cancer, sinonasal cancer, or non-small cell lung cancer. In some embodiments, the cancer is non-small cell lung cancer, prostate cancer, head and neck cancer, breast cancer, colorectal cancer, or glioblastoma In some embodiments, the cancer is non-small cell lung cancer. In some embodiments, the cancer is prostate cancer. In some embodiments, the cancer is head and neck cancer. In some embodiments, the cancer is breast cancer. In some embodiments, the cancer is colorectal cancer. In some embodiments, the cancer is glioblastoma. In some embodiments, the glioblastoma is pediatric bithalamic glioma.

In some embodiments, the cancer in the subject comprises an EGFR mutation i.e., the cancer is characterized by an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR (or D770_N771insSVD EGFR), 770insNPG EGFR (or D770_N771insNPG EGFR), 770insGT EGFR, 770insGF EGFR, 770insG EGFR, 771insH EGFR, 771insN EGFR, 772insNP EGFR, 773insNPH EGFR (or H773insNPH EGFR), 773insH EGFR, 773insPH EGFR, EGFRvii, EGFRviii, A767_dupASV EGFR, 773insAH EGFR, M766_A767insAI EGFR, and any combination thereof. In some embodiments, the EGFR mutation is del19/T790M EGFR or L858R/T790M EGFR. In some embodiments, the EGFR mutation is del19/T790M EGFR. In some embodiments, the EGFR mutation is L858R/T790M EGFR. In some embodiments, the cancer is characterized by an EGFR mutation. In some embodiments, the cancer is characterized by an insertion in exon 20. In some embodiments, the cancer is characterized by an EGFR exon 18 G719X or exon 21 L861Q mutation. In some embodiments, the mutation is S768I EGFR.

The presence of CNS metastases, including brain and leptomeningeal lesions, can cause significant morbidity and has been associated with poorer outcomes for patients with NSCLC, including EGFR mutated disease. Management of brain metastases depends upon the extent of the disease and need for emergent treatment. For patients who do not need immediate surgical therapy, initial treatment can consist of radiation therapy, preferably with stereotactic radiosurgery rather than whole brain radiation. Radiation can be associated with cognitive decline due to radiation-induced necrosis, which can have a significant impact on quality of life as patients have longer survival due to improved therapies. While chemotherapy has some activity, outcomes tend to be inferior to those achieved with radiation. For patients with common EGFR mutations, treatment with osimertinib is an option rather than radiation therapy, although this approach has not been formally evaluated in a randomized trial. There is a need for an EGFR Ex20ins-targeted TKI that is highly CNS penetrant. In some aspects, provided herein is a method of treating CNS (or brain) metastases associated with a cancer characterized by mutant EGFR in a subject in need thereof, comprising administered to the subject a therapeutically effective amount of a solid form or salt form of Compound (I). In some aspects, provided herein is a method of treating asymptomatic brain metastases.

In another aspect, provided herein is a method of treating inflammatory disease in a subject in need thereof, comprising administering to the subject a therapeutically effective amount of a solid form or a salt form of Compound (I). Also described herein is the use of the solid forms and salt forms of Compound (I) described herein for treating inflammatory diseases associated with EGFR del19/T790M activation. Also described herein is the use of the solid forms and salt forms of Compound (I) described herein for treating inflammatory diseases associated with EGFR L858R/T790M activation.

In some embodiments, the inflammatory disease is psoriasis, eczema, or atherosclerosis. In some embodiments, the inflammatory disease is psoriasis. In some embodiments, the inflammatory disease is eczema. In some embodiments, the inflammatory disease is atherosclerosis.

In some embodiments, the inflammatory disease in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is selected from del19/T790M EGFR, L858R/T790M EGFR, L858R EGFR, L861Q EGFR, S768I EGFR, G719X EGFR, 763insFQEA EGFR, 767insTLA EGFR, 769insASV EGFR, 769insGE EGFR, 770insSVD EGFR (or D770_N771insSVD EGFR), 770insNPG EGFR (or D770_N771insNPG EGFR), 770insGT EGFR, 770insGF EGFR, 770insG EGFR, 771insH EGFR, 771insN EGFR, 772insNP EGFR, 773insNPH EGFR (or H773insNPH EGFR), 773insH EGFR, 773insPH EGFR, EGFRvii, EGFRviii, A767_dupASV EGFR, 773insAH EGFR, M766_A767insAI EGFR, and any combination thereof. In some embodiments, the EGFR mutation is del19/T790M EGFR or L858R/T790M EGFR. In some embodiments, the EGFR mutation is del19/T790M EGFR. In some embodiments, the EGFR mutation is L858R/T790M EGFR. In some embodiments, the mutation is EGFR exon 18 G719X or exon 21 L861Q. In some embodiments, the mutation is S768I EGFR.

### Administration and Pharmaceutical Composition

In certain embodiments, the solid form or a salt form of Compound (I) is administered as a pure chemical. In other embodiments, the solid form or a salt form of Compound (I) is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, PA (2005)).

Provided herein is a pharmaceutical composition comprising at least one solid form or a salt form of Compound (I) together with one or more pharmaceutically acceptable carriers. One embodiment provides a pharmaceutical composition comprising a solid form or a salt form of Compound (I) and a pharmaceutically acceptable excipient.

In certain embodiments, the solid form or a salt form of Compound (I) is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21st Ed. Mack Pub. Co., Easton, PA (2005)).

The dose of the pharmaceutical composition comprising at least one solid form or a salt form of Compound (I) differ, depending upon the patient's condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the active ingredient(s) in an amount sufficient to provide therapeutic benefit (e.g., an improved clinical outcome), or a lessening of symptom severity. Optimal doses are generally determined using experimental models and/or clinical trials. The optimal dose depends upon the body mass, weight, or blood volume of the patient.

The following examples are intended to be illustrative and are not intended to be limiting in any way to the scope of the disclosure.

### EXPERIMENTAL

### Instruments and Methods

Different instruments and methods were used for testing and characterizing different batches of materials. For example, XRPD peaks described in Tables 1, 2, 3, 4, 5a, 6a, 7a, 8a, 9a, 10a, 11a, 12a, 13a, 14a, 15a and 16a were collected using XRPD method 1 and XRPD peaks described in Tables 5b, 6b, 7b, 8b, 9b, 10b, 11b, 12b, 12c, 13b, 13c, 13d, 14b, 15b and 16b were collected using XRPD method 2.

### X-Ray Powder Diffraction (XRPD) Method 1

Instrument: Panalytical Empyrean
Parameters: X-Ray tube Cu (Kα radiation); tube voltage 40 kV; tube current 15 mA
Scanning range: 2 to 40 2θ (degree)
Step size: 0.01 degree
Scanning speed: 1.31 degree (2θ) per minute

### X-Ray Powder Diffraction (XRPD) Method 2

Using zero-background sample holder designed for small sample loadings, ensure the mirrored surface must not be scratched, the sample should be loaded into the dimple in the of the sample holder. For sample preparation, the sample mount level must be flushed with the top of the recess the sample it meant to inhabit. Use a kimwipe with some solvent (i.e. Methanol) on it to wipe clean the sample holder. Use a spatula to transfer a small amount of powder onto the sample holder and push all surrounding powder material on the mirror surface onto the middle dimple. Ensure not to scratch the mirror surface of the sample holder with the spatula. Use a clean glass slide, gently push down on the powder sample in the dimple area of the sample holder and rotate the slide to flatten the sample. Clean the mirror surface outside of the sample dimple area with a kimwipe before placing the sample holder into the XRPD chamber for analysis.

XRPD was performed using a Bruker D8 Advance equipped with LYNXEYE detector in reflection mode (i.e. Bragg-Brentano geometry). Samples were prepared on Si zero-return wafers as described above. The parameters for XRPD methods used are listed below:

| Parameter | Regular scan | High resolution scan |
|---|---|---|
| X-ray wavelength | Cu Kαl, 1.540598 Å | Cu Kαl, 1.540598 Å |
| X-ray tube setting | 40 kV, 40 mA | 40 kV, 40 mA |
| Slit condition | 0.6 mm div. + 2.5° soller | 0.6 mm div. + 2.5° soller |
| Scan mode | Step | Step |
| Scan range (°2θ) | 4-30 | 4-40 |
| Step size (°2θ) | 0.03 | 0.02 |
| Dwell time (s/step) | 0.23 | 0.9 |
| Spin | Yes (0.5 Hz) | Yes (0.5 Hz) |

### Thermogravimetric Analysis (TGA)

Instrument: TA Instruments Discovery TGA
Parameters: Ramp 10° C. per minute, 25 to 300° C., 50 mL/min N₂ sweep

### Differential Scanning Calorimetry (DSC) Method 1

Instrument: TA Instruments Discovery DSC
Parameters: Ramp 10° C. per minute, up to 300° C.

### Differential Scanning Calorimetry (DSC) Method 2

DSC was performed using a TA Discovery DSC. The sample (1-5 mg) was weighed directly in a 40 μL hermetic aluminum pan with a pinhole and analyzed according to the parameters below:

Parameters

| | |
|---|---|
| Method | Ramp |
| Sample size | 1-5 mg |
| Heating rate | 10.0° C./min |
| Temperature range | 30 to 300° C. |
| Method gas | N₂ at 50.00 mL/min |

### Simultaneous Thermogravimetric Analysis and Differential Scanning Calorimetry (TGA and DSC):

TGA and DSC were performed on the same sample simultaneously using a Mettler Toledo TGA/DSC3+. Protective and purge gas was nitrogen at a flowrate of 20-30 mL/min and 50-100 mL/min, respectively. The desired amount of sample (5-10 mg) was weighed directly in a hermetic aluminum pan with pinhole and analyzed according to the parameters below:

Parameters

| | |
|---|---|
| Method | Ramp |
| Sample size | 5-10 mg |
| Heating rate | 10.0° C./min |
| Temperature range | 30 to 300° C. |

### Dynamic Vapor Sorption (DVS) Method 1

Dynamic vapor sorption was performed with a TA Instruments Q5000SA DVS at 25° C. under nitrogen blow. Approximately 10-15 mg of material was used. Samples were analyzed using methods below:

For anhydrate:
0% RH to 90% RH at 10% RH
90% RH to 0% RH at 10% RH
   For anhydrate:
40% RH to 90% RH at 10% RH
90% RH to 0% RH at 10% RH

### Dynamic Vapor Sorption (DVS) Method 2

DVS was performed using a Q5000SA. The sample (5-15 mg) was loaded into a metallic quartz sample pan, suspended from a microbalance, and exposed to a humidified stream of nitrogen gas. Weight changes were relative to a matching empty reference pan opposite the sample, suspended

from the microbalance. The sample was held for a minimum of 10 min at each level and only progressed to the next humidity level if there was <0.002% change in weight between measurements (interval: 5 s) or 45 min had elapsed (for 5-65% RH) or 2 h had elapsed (for 80 and 95% RH). The following program was used:
1—Equilibration at 50% RH
2—50% to 5%. (50%, 35%, 20%, and 5%)
3—5% to 95% (5%, 20%, 35%, 50%, 65%, 80%, and 95%)
4—95% to 5% (95%, 80%, 65%, 50%, 35%, 20%, and 5%)
5—5% to 50% (5%, 20%, 35%, and 50%)

### Polarized Light Microscopy (PLM)

Instrument: Nikon Eclipse Ci POL
Camera: Nikon DS-Fi3
Software: Nikon NIS Elements

### Microscopy:

Optical microscopy was performed using a Zeiss AxioScope A1 digital imaging microscope equipped with 2.5×, 10×, and 40× objectives and polarizer. Images were captured through a built-in Axiocam 105 digital camera and processed using ZEN 2 (blue edition) software provided by Zeiss.

### High Performance Liquid Chromatography (HPLC):

HPLC was conducted using an Agilent 1220 Infinity 2 LC equipped with diode array detector (DAD). Flow rate range of the instrument is 0.2-5.0 mL/min, operating pressure range is 0-600 bar, temperature range is 5° C. above ambient to 60° C., and wavelength range is 190-600 nm

The HPLC method used in this study is shown below:

Parameters

| Mobile phase A | 0.05% TFA in distilled water | | |
|---|---|---|---|
| Mobile phase B | 0.05% TFA in ACN | | |
| Diluent | ACN:water (7:3 vol.) | | |
| Injection volume | 5 μL | | |
| Monitoring wavelength | 210 nm | | |
| Column | Waters Xbridge C-18, 4.6 × 150 mm, 3.5 μm | | |
| Column temperature | not controlled | | |
| | Time (min) | % B | Flow rate (mL/min) |
| Gradient method | 0 | 5 | 1.0 |
| | 1 | 5 | 1.0 |
| | 16 | 85 | 1.0 |
| | 19.5 | 85 | 1.0 |
| | 19.6 | 5 | 1.0 |
| | 23 | 5 | 1.0 |

### Proton Nuclear Magnetic Resonance (¹H NMR) Spectroscopy:

¹H NMR was performed on Bruker Avance 300, 400 and 500 MHz spectrometers. Solids were dissolved in 0.75 mL deuterated solvent in a 4 mL vial, transferred to an NMR tube (Wilmad 5 mm thin wall 8″ 200 MHz, 506-PP-8) and analyzed according to the following parameters:

Parameters - Bruker Avance 300

| Instrument | Bruker Avance 300 MHz spectrometer | |
|---|---|---|
| Temperature | 300 | K |
| Probe | 5 mm PABBO BB-1H/DZ-GRD Z104275/0170 | |
| Number of scans | 16 | |
| Relaxation delay | 1.000 | s |
| Pulse width | 14.2500 | μs |
| Acquisition time | 2.9999 | s |
| Spectrometer frequency | 300.15 | MHz |

| | |
|---|---|
| Nucleus | ¹H |

Parameters - Bruker Avance 400

| Instrument | Bruker Avance 400 MHz Neo Nanobay spectrometer | |
|---|---|---|
| Temperature | 298 | K |
| Probe | Z163739_0636 (PI HR-BBO400S1-BBF/H/D-5.0-Z SP) | |
| Number of scans | 32 | |
| Relaxation delay | 1.0000 | s |
| Pulse width | 7.7000 | μs |
| Acquisition time | 3.9977 | s |
| Spectrometer frequency | 400.30 | MHz |

| | |
|---|---|
| Nucleus | ¹H |

Parameters - Bruker Avance 500

| Instrument | Bruker Avance 500 MHz spectrometer | |
|---|---|---|
| Temperature | 300 | K |
| Probe | 5 mm PABBO BB-1H/D Z-GRD Z113652/0159 | |
| Number of scans | 32 | |
| Relaxation delay | 1.000 | s |
| Pulse width | 14.0000 | μs |
| Acquisition time | 3.2506 | s |
| Spectrometer | 500.13 | MHz |
| frequency | | |

| | |
|---|---|
| Nucleus | ¹H |

### Example 1: Preparation of Compound (I)

### Synthesis of N-(4-fluoro-3-((2-((1-methyl-1H-pyrazol-4-yl)amino)-5-(4-(trifluoro methyl)phenyl)pyrimidin-4-yl)amino)phenyl)acrylamide (Compound I)

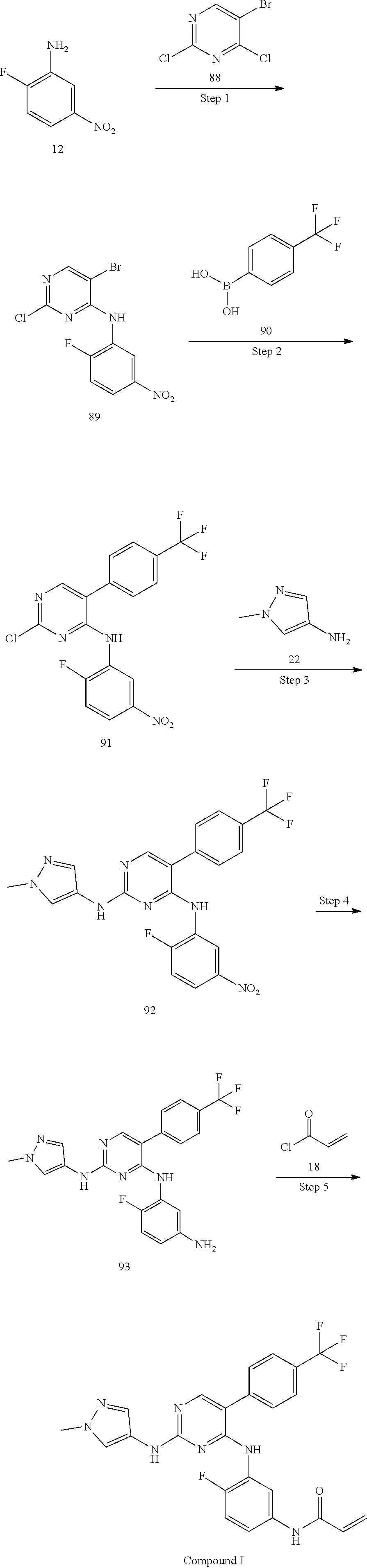

### Step 1: Synthesis of 5-bromo-2-chloro-N-(2-fluoro-5-nitrophenyl)pyrimidin-4-amine (89)

To an ice cold solution of 2-fluoro-5-nitroaniline (12) (1.0 eq) in tetrahydrofuran was added sodium hydride (60% dispersion in mineral oil, 3.0 eq) portion-wise. The resulting reaction mixture was stirred at room temperature for 30 minutes and followed by the addition of 2,4-dichloro-5-bromopyrimidine (88) (1.0 eq). The resulting reaction mixture was heated at 60° C. for 16 hours. After completion (TLC monitoring), quenched with ice, extracted with ethyl acetate (3 times). The combined organic layers were washed with water, brine, dried over anhydrous sodium sulfate, filtered and concentrated under reduced pressure. The crude was purified by combiflash eluted with 40% ethyl acetate in hexane to get (89) as pale yellow solid (1.3 g, Yield: 44.24%). MS: [M+H]⁺ 346.97.

### Step 2: Synthesis of 2-chloro-N-(2-fluoro-5-nitrophenyl)-5-(4-(trifluoromethyl)phenyl) pyrimidin-4-amine (91)

To a solution of halo derivative (89) (1.0 eq) and respective boronate acid/ester derivative (90) (1.1 eq) in N,N-dimethylformamide:water (4:1) was added sodium carbonate or sodium bicarbonate (2.0 eq). The resulting reaction mixture was degassed under argon atmosphere for 15 minutes, followed by addition of tetrakis(triphenylphosphine)palladium(0) (0.1 eq). The resulting reaction mixture was heated at 90° C. for 16 hours. After completion of reaction (TLC monitoring), the reaction mixture was cooled to room temperature, water was added and extracted with ethyl acetate (3 times). The combined organic layers were washed with brine, dried over anhydrous sodium sulfate, filtered and concentrated under reduced pressure. The crude was purified by combiflash eluted with 35% ethyl acetate in hexane to get desired product (91) as light yellow solid (700 mg; Yield: 50.12%). MS: [M+H]⁺ 413.10.

### Step 3: Synthesis of N4-(2-fluoro-5-nitrophenyl)-N2-(1-methyl-1H-pyrazol-4-yl)-5-(4-(trifluoromethyl)phenyl)pyrimidine-2,4-diamine (92)

To an ice-cold solution of chloro compound (91) (1.0 eq) in isopropanol was added amine (22) (1.2 eq) and trifluoroacetic acid (2.0 eq). The reaction mixture was heated at 110° C. for 16 hours. After completion of the reaction (TLC monitoring), the reaction mixture was concentrated under reduced pressure, added saturated solution of sodium bicarbonate and extracted with dichloromethane (3 times). The combined organic layers were washed with brine solution, dried over anhydrous sodium sulfate and evaporated under reduced pressure. The crude was purified by combiflash eluted with 1% methanol in dichloromethane to get desired product (92) as pale yellow solid (500 mg; Yield: 70.24%). MS: [M+H]⁺ 474.09.

### Step 4: Synthesis of N4-(5-amino-2-fluorophenyl)-N2-(1-methyl-1H-pyrazol-4-yl)-5-(4-(trifluoromethyl)phenyl)pyrimidine-2,4-diamine (93)

To an ice cold solution of nitro derivative (92) (1.0 eq) in methanol:tetrahydrofuran:water (2:2:1) were added zinc-dust or iron powder (5 eq) and ammonium chloride (5 eq). The resultant reaction mixture was stirred at room temperature for 2 hours. After completion of reaction (TLC monitoring), reaction mixture passed through celite bed washed with 5% methanol in dichloromethane. The filtrate was washed with water, brine, dried over anhydrous sodium sulfate, filtered and concentrated to dryness to get the desired product (93) as semi solid (350 mg; Yield: 74.78%). MS: [M+H]⁺ 444.11.

### Step 5: Synthesis of N-(4-fluoro-3-((2-((1-methyl-1H-pyrazol-4-yl)amino)-5-(4-(trifluoromethyl)phenyl)pyrimidin-4-yl)amino)phenyl)acrylamide (Compound I)

To a solution of amino compound (93) (1.0 eq) in dichloromethane:tetrahydrofuran (1:1) was cooled to −40° C. followed by triethylamine (3-5 eq) and acryloyl chloride (1.0 eq) were added. The mixture was stirred at the same temperature for 2 hours. After completion of reaction (monitored by TLC), added water and extracted with dichloromethane (3 times). The combined organic layers washed with brine, dried over anhydrous sodium sulfate, filtered and concentrated under reduced pressure. The crudes were purified by Prep-HPLC purification to to obtain Compound I as off white solid (30 mg, Yield: 13.33%). ¹H NMR (400 MHz, DMSO-d₆): δ 10.21 (bs, 1H), 9.24 (bs, 1H), 8.53 (bs, 1H), 7.99 (s, 1H), 7.71-7.81 (m, 5H), 7.57 (s, 1H), 7.08-7.16 (m, 3H), 6.37-6.44 (m, 1H), 6.21-6.26 (m, 1H), 5.74 (d, J=8.4 Hz, 1H), 3.54 (s, 3H). LCMS: [M+H]⁺ 498.35.

### Example 2: Preparation of Mesylate Salt of Compound (I) Form A

About 30 mg of Compound (I) free base was dissolved in 1.7 mL acetone (or 7.5 mL ethyl acetate) to obtain a clear solution. Methanesulfonic acid in a 1:1 molar charge ratio was added to the free base solution and the mixture was stirred at room temperature (rt) for 2 days. Precipitate was isolated by centrifugation, cooling to 5° C., or slow evaporation to obtain a product. Alternatively to stirring at rt, the mixture could be heated to 40° C. and stirred overnight and seeded with Form A crystals.

XRPD analysis confirmed that the obtained product is Form A. Mesylate Form A was confirmed to be an anhydrate of a mono-mesylate salt.

### Example 3: Preparation and Characterization of Mesylate Salt of Compound (I) Form B

### General Synthesis for Mesylate Form B:

Mesylate Form B can generally be obtained by slurrying or dissolving Compound (I) in acetone (e.g., 10-30 vol., 10-25 vol., 15-25 vol. etc.) at elevated temperature (e.g., between 40° C. and 70° C., between 40° C. and 65° C., between 45° C. and 55° C. or between 50° C. and 60° C., etc.) or reflux. The solution was then cooled slightly, and a small portion of methanesulfonic acid (e.g., 0.1-0.5 molar equivalent to compound (I)) was then charged, which can result in dissolution. Seeds (Mesylate Form B) were then charged, and once the seed bed formed, the remainder of the methanesulfonic acid was charged. Alternatively, seed crystals were not used and the remainder of the methanesulfonic acid was charged. Following an optional hold period (e.g., 10 minutes to 5 hours, 10-120 minutes, 30-100 minutes, etc.), the slurry was then cooled and held at ambient or lower temperature (e.g., between 5° C. and 25° C., between 5° C. and 15° C., between 15° C. and 25° C., etc.) and then filtered and washed with acetone. Drying the product at elevated temperature yielded Mesylate Form B (e.g., between 40° C. and 70° C., between 40° C. and 65° C., between 45° C. and 55° C. or between 50° C. and 60° C., etc.). In some embodiments, a mixture of acetone and small amount of water (e.g., 1-5%) can also be used for slurrying or dissolving Compound (I) instead of acetone.

### Example 3a

2.595 g of Compound (I) freebase was heated to reflux in 20 vol. of acetone. 0.3 eq. of MSA was then charged then cooled to 42° C. Seed (0.1% mesylate pattern B) was then charged and stirred for 40 min. MSA (0.8 eq.) was then charged and the slurry was stirred at 42° C. for 1 h. Alternatively, seed crystals were not added and the remaining MSA (0.8 eq.) was added in three equal parts and the slurry was stirred at 42° C. for 1 h, After cooling to 20° C. for 1 h, the slurry was then stirred for 1 h, then filtered and washed with 3.0 vol. of acetone. The solids were dried at 50° C. under vacuum overnight. The yield was 2.54 g (86%) as Mesylate Form B.

### Example 3b

500 mg of Compound (I) free base was dissolved in 20 mL acetone and heated to 50° C. to obtain a clear solution. 75 μL (1.1 M. eq) of methanesulfonic acid were added to the free base solution. Seed crystals (Form B) were added and the mixture was stirred at room temperate for 2 days. Isolated solids weighed 507 mg (89% yield) as Mesylate Form B.

XRPD analysis confirmed that the isolated solid is Form B. The product was characterized by HPLC, PLM, TGA/DSC (FIG. 2B) and 1H-NMR. Mesylate Form B was confirmed to be an anhydrate of a mono-mesylate salt with irregular shape plate-like morphology.

TGA analysis demonstrated negligible mass loss up to 150° C. (FIG. 2B). The DSC demonstrated an endotherm with an onset of 247.8° C.

DVS shows 2.47% weight gain at 80% RH, suggesting it may be hygroscopic, but no crystal form change post DVS was observed.

### Example 4: Preparation and Characterization of Mesylate Salt of Compound (I) Form H

Mesylate Form H was prepared through slow-cooling crystallization in methanol:acetone (1:1 vol). Approximately 300 mg of Mesylate Form B was weighed into a 4 mL vial. A solvent mixture of methanol and acetone (4.5 vol) was then added incrementally at 50° C. until dissolution. The solution was cooled at 5° C. per hour, while mixing, to 5° C. using a cooling block with a programmable chiller. The slurry was filtered and washed with solvent (2×0.5 vol), then dried under vacuum at 50° C. overnight. A yield of 0.166 g (49 mol %) was obtained as Mesylate Form H.

Mesylate Form H was produced at approximately 0.200 g scale though a short-term slurry of Mesylate Form B in ethanol at 50° C. Seeding was carried out with Mesylate Form H. A yield of 0.159 g (79.5 w/w %) was obtained. Based on the characterization performed, Mesylate Form H was determined to be anhydrous.

Slurry competition experiments among all anhydrates of mesylate salt including Form A, Form B, and Form H were conducted to determine the relative stability using THF as solvent at 25° C. and using isopropyl alcohol (IPA) as solvent at 25° C. and 60° C. At the end of the study, the 3 salt forms converted to Form H in THF at 25° C. (in 3 days) and in IPA at both 25° C. (in 3 days) and 60° C. (in 1 day). Therefore, in THF and IPA, Mesylate Form H is a thermodynamically more stable form at both RT and 60° C.

Mesylate Form H was confirmed to be an anhydrate of a mono-mesylate salt with needle-like morphology.

TGA analysis demonstrated a mass loss of 0.409 wt. % up to 150° C. (FIG. 3B). The DSC demonstrated two endothermic and exothermic events; a large endotherm with an onset of 220.54° C., and a small exotherm with an onset of 239.42° C. (FIG. 3B).

To understand the hygroscopicity of Mesylate Form H, dynamic vapor sorption (DVS) was employed to measure the mass change as a function of relative humidity at 25° C. Anhydrate Mesylate Type H was equilibrated at 0% RH to remove the adsorbed moisture or residual solvent before analysis. The results indicated, Mesylate Form H showed a water uptake of 1.76% at 25° C./80% RH, suggesting Mesylate Form H is slightly hygroscopic. No form change was observed for sample after DVS evaluation.

### Example 5: Preparation and Characterization of Mesylate Salt of Compound (I) Form I

Mesylate Form I was prepared through slow-cooling crystallization in ethanol:water (9:1 vol). Approximately 300 mg of Mesylate Form B was weighed into a 4 mL vial. Solvent (9 vol) was then added incrementally at 50° C. until dissolution. The solution was cooled at 5° C. per hour, while mixing, to 5° C. using a cooling block with a programmable chiller. The slurry was filtered and washed with solvent (2×0.5 vol), then dried under vacuum at 50° C. overnight. A yield of 0.164 g (48 mol %) was obtained as Mesylate Form I.

Mesylate Form I was confirmed to be a hydrate from solid state characterization results.

TGA analysis demonstrated a mass loss of 1.998 wt. % up to 150° C. (FIG. 4B). The DSC demonstrated three endothermic and exothermic events; a small endotherm with an onset of 33.16° C., a large endotherm with an onset of 210.43° C., and a small exotherm with an onset of 229.09° C. (FIG. 4B).

DVS results for Mesylate Form I showed a water uptake of 0.54% from 30% RH to 80% RH at 25° C., suggesting Mesylate Form I is slightly hygroscopic. No form change was observed for sample after DVS evaluation.

### Example 6: Preparation and Characterization of Crystalline Freebase Form A of Compound (I)

It was characterized by X-ray powder diffraction (XRPD), polarized light microscopy (PLM), thermogravimetric analysis (TGA), differential scanning calorimetry (DSC) and high performance liquid chromatography (HPLC). The characterization results indicated that the starting material was crystalline, defined as free base Form A, and the crystal form was further identified to be an anhydrate with rod-like morphology.

DVS results for free base Form A showed a water uptake of 5.23% at 80% RH. No form change was observed for sample after DVS evaluation.

### Example 7: Preparation and Characterization of Crystalline Freebase Form B of Compound (I)

About 30 mg of Compound (I) was dissolved in 1.7 mL of acetone. The mixture was stirred for 2 days at room temperature. Free base Form B of Compound (I) was obtained which was confirmed by XRPD, DSC and TGA.

### Example 8: Pharmacokinetics of Mesylate Salt and Freebase Form of Compound (I) Following Oral (PO) Administration to Male Beagle Dogs

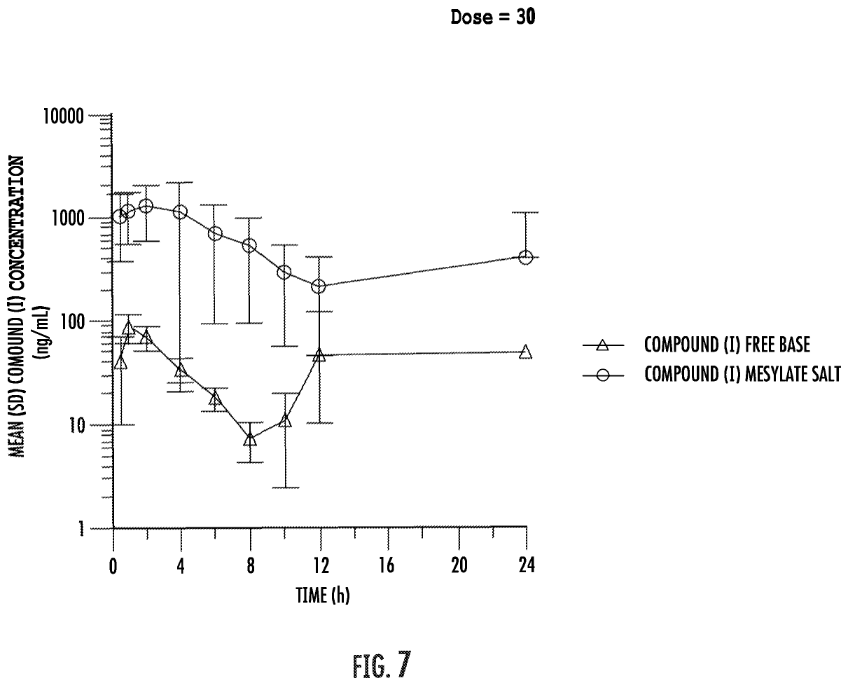

Male beagle dogs (3 in each treatment group) were administered orally 30 mg/kg of Compound (I) free base or mesylate salt of Compound (I) in a dose equivalent to 30 mg/kg of free base. The free base and the mesylate salt were suspended/dissolved in an appropriate volume of vehicle solution containing 0.5% hydroxypropyl methylcellulose (w/v) with 0.2% Tween 80 (v/v) in deionized water. The PO doses were administered into the stomach via syringe and gavage tube followed by a 5 mL water flush. Dose volumes were adjusted for the weight of the animal on the morning of dose administration. Blood samples were collected at 0, 0.5, 1, 2, 4, 6, 8, 10, 12, and 24 h postdose from an accessible vein with preference to the jugular vein. Blood samples were kept on ice until processing. All blood samples were centrifuged within 1 h of collection at 3200 RPM for 10 min at 5° C. Plasma samples were directly transferred into cluster tubes and stored at −20°±5° C. until shipment. All animals appeared normal at dose administration and all of the blood collection time points. Animals were fasted on the evening prior to dosing with food returned at 4 h postdose. As shown in FIG. 7, the mesylate salt provided higher in vivo exposure compared to the free base form.

1. A mesylate salt of Compound (I) represented by the following structural formula: 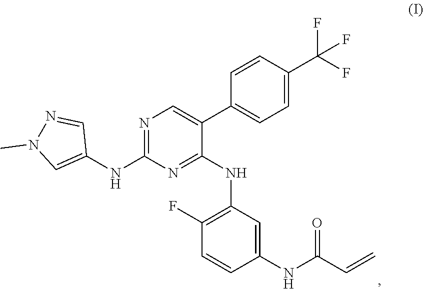 [SMILES: C=CC(=O)Nc1ccc(F)c(Nc2nc(Nc3cnn(C)c3)ncc2-c2ccc(C(F)(F)F)cc2)c1]
wherein the molar ratio between Compound (I) and methanesulfonic acid is 1:1.

2. The mesylate salt of claim 1, wherein the mesylate salt is a crystalline salt.

3. The mesylate salt of claim 2, wherein the crystalline mesylate salt is crystalline Form A characterized by an X-ray powder diffraction pattern comprising at least three, at least four or at least five peaks selected from 5.9°, 7.2°, 11.9°, 12.1°, 19.3° and 20.1°±0.2° in 2θ.

4. The mesylate salt of claim 3, wherein the crystalline mesylate salt is crystalline Form A characterized by an X-ray powder diffraction pattern comprising peaks at 5.9°, 7.2°, 11.9°, 12.1°, 19.3° and 20.1°±0.2° in 2θ.

5. The mesylate salt of claim 4, wherein said Form A characterized by an X-ray powder diffraction pattern comprising peaks at 5.9°, 7.2°, 11.9°, 12.1°, 13.4°, 19.3°, 20.1°, 21.7°, 24.1°, and 27.6°±0.2° in 2θ.

6. The mesylate salt of claim 4 or 5, wherein said Form A characterized by an X-ray powder diffraction pattern comprising peaks at 5.9°, 7.2°, 11.9°, 12.1°, 13.4°, 14.6°, 15.4°, 19.3°, 20.1°, 21.7°, 22.9°, 23.8°, 24.1° and 27.6°±0.2° in 2θ.

7. The mesylate salt of any one of claims 3-6, wherein said Form A characterized by an X-ray powder diffraction pattern substantially similar to FIG. 1A.

8. The mesylate salt of any one of claims 3-7, wherein said Form A is further characterized by a differential scanning calorimeter (DSC) thermogram comprising an endotherm onset at 202.8° C.±2° C., and an exotherm onset at 206.9° C.±2° C.

9. The mesylate salt of any one of claims 3-8, wherein said Form A is further characterized by a thermogravimetric analysis (TGA) substantially similar to FIG. 1B.

10. The mesylate salt of claim 2, wherein the crystalline mesylate salt is crystalline Form B characterized by an X-ray powder diffraction pattern comprising at least three or at least four peaks selected from 18.5°, 19.8°, 20.8°, 22.4° and 24.9°±0.2° in 2θ.

11. The mesylate salt of claim 2, wherein the crystalline mesylate salt is crystalline Form B characterized by an X-ray powder diffraction pattern comprising peaks at 18.5°, 19.8°, 20.8°, 22.4° and 24.9°±0.2° in 2θ.

12. The mesylate salt of claim 11, wherein said Form B is characterized by an X-ray powder diffraction pattern comprising peaks at 8.9°, 11.3°, 18.5°, 19.8°, 20.8°, 21.4°, 22.4°, 24.9°, and 25.9°±0.2° in 2θ.

13. The mesylate salt of claim 2, wherein the crystalline mesylate salt is crystalline Form B characterized by an X-ray powder diffraction pattern comprising at least three or at least four peaks selected from 18.4°, 19.7°, 21.3°, 22.4°, 24.8°±0.2° in 2θ.

14. The mesylate salt of claim 13, wherein the crystalline mesylate salt is crystalline Form B characterized by an X-ray powder diffraction pattern comprising peaks at 18.4°, 19.7°, 21.3°, 22.4°, 24.8°±0.20 in 2θ.

15. The mesylate salt of claim 13, wherein the crystalline mesylate salt is crystalline Form B characterized by an X-ray powder diffraction pattern comprising peaks at 8.8°, 11.3°, 18.4°, 19.7°, 20.6°, 20.8°, 21.3°, 22.0°, 22.4° and 24.8°±0.20 in 2θ.

16. The mesylate salt of claim 13, wherein the crystalline mesylate salt is crystalline Form B characterized by an X-ray powder diffraction pattern comprising peaks at 5.4°, 6.6°, 7.6°, 8.8°, 11.3°, 11.8°, 12.3°, 13.6°, 15.2°, 15.4°, 16.4°, 16.7°, 17.1°, 18.0°, 18.4°, 19.5°, 19.7°, 20.6°, 20.8°, 21.3°, 21.6°, 22.0°, 22.4°, 22.7°, 23.7°, 24.5°, 24.8°, 25.4°, 25.7°, 25.8°, 26.5°, 27.5°, 29.8°, 30.8°, 31.2°, 35.0°, 35.3° and 36.3°±0.2° in 2θ.

17. The mesylate salt of claim 2, wherein said Form B is characterized by an X-ray powder diffraction pattern substantially similar to FIG. 2A.

18. The mesylate salt of any one of claims 9-17, wherein said Form B is further characterized by a differential scanning calorimeter (DSC) thermogram comprising an endotherm onset at 247.8° C.±2° C.

19. The mesylate salt of any one of claims 9-18, wherein said Form B is further characterized by a thermogravimetric analysis (TGA) substantially similar to FIG. 2B.

20. The mesylate salt of claim 2, wherein the crystalline mesylate salt is crystalline Form H characterized by an X-ray powder diffraction pattern comprising at least three, at least four or at least five peaks selected from 13.7°, 19.1°, 20.0°, 21.5°, 21.9°, and 23.4°±0.2° in 2θ.

21. The mesylate salt of claim 20, wherein the crystalline mesylate salt is crystalline Form H characterized by an X-ray powder diffraction pattern comprising peaks at 13.7°, 19.1°, 20.0°, 21.5°, 21.9°, and 23.4°±0.2° in 2θ.

22. The mesylate salt of claim 21, wherein said Form H is characterized by an X-ray powder diffraction pattern comprising peaks at 8.1°, 10.1°, 11.7°, 13.7°, 19.1°, 20.0°, 20.8°, 21.5°, 21.9°, and 23.4°±0.2° in 2θ.

23. The mesylate salt of any one of claims 20-22, wherein said Form H is characterized by an X-ray powder diffraction pattern comprising peaks at 8.1°, 10.1°, 10.7°, 11.7°, 13.7°, 14.6°, 19.1°, 20.0°, 20.8°, 21.5°, 21.9°, 23.4°, and 24.7°±0.2° in 2θ.

24. The mesylate salt of claim 2, wherein the crystalline mesylate salt is crystalline Form H characterized by an X-ray powder diffraction pattern comprising at least three or at least four peaks selected from 19.1°, 20.0°, 21.5°, 21.9° and 23.4°±0.2° in 2θ,

25. The mesylate salt of claim 24, wherein the crystalline mesylate salt is crystalline Form H characterized by an X-ray powder diffraction pattern comprising peaks at 19.1°, 20.0°, 21.5°, 21.9° and 23.4°±0.2° in 2θ.

26. The mesylate salt of claim 24, wherein the crystalline mesylate salt is crystalline Form H characterized by an X-ray powder diffraction pattern comprising peaks at 11.7°, 13.6°, 14.6°, 19.1°, 20.0°, 20.8°, 21.5°, 21.9°, 23.4°, 24.6°±0.2° in 2θ.

27. The mesylate salt of claim 24, wherein the crystalline mesylate salt is crystalline Form H characterized by an X-ray powder diffraction pattern comprising peaks at 6.9°, 8.1°, 10.1°, 10.7°, 11.7°, 12.3°, 12.7°, 13.2°, 13.6°, 13.9°, 14.6°, 14.9°, 16.2°, 16.8°, 17.1°, 17.9°, 18.1°, 18.8°, 19.1°, 19.7°, 20.0°, 20.6°, 20.8°, 21.0°, 21.5°, 21.9°, 22.1°, 22.8°, 23.2°, 23.4°, 24.1°, 24.3°, 24.6°, 25.4°, 25.6°, 26.4°, 26.8°, 27.1°, 27.8°, 28.2°, 29.0°, 29.6°, 30.0°, 31.2°, 31.8°, 32.1°, 32.6°, 33.3°, 34.0°, 34.3°, 34.8°, 35.6°, 37.3°, and 38.7°±0.2° in 2θ.

28. The mesylate salt of any one of claims 20-27, wherein said Form H is characterized by an X-ray powder diffraction pattern substantially similar to FIG. 3A.

29. The mesylate salt of any one of claims 20-28, wherein said Form H is further characterized by a differential scanning calorimeter (DSC) thermogram comprising an endotherm onset at 220.5° C.±2° C., and an exotherm onset at 239.4° C.±2° C.

30. The mesylate salt of any one of claims 20-29, wherein said Form H is further characterized by a thermogravimetric analysis (TGA) substantially similar to FIG. 3B.

31. The mesylate salt of claim 2, wherein the crystalline mesylate salt is crystalline Form I characterized by an X-ray powder diffraction pattern comprising at least three or at least four peaks selected from 11.0°, 18.7°, 20.6°, 22.2°, and 24.4°±0.2° in 2θ.

32. The mesylate salt of claim 31, wherein the crystalline mesylate salt is crystalline Form I characterized by an X-ray powder diffraction pattern comprising peaks at 11.0°, 18.7°, 20.6°, 22.2°, and 24.4°±0.2° in 2θ.

33. The mesylate salt of claim 31 or 32, wherein said Form I is characterized by an X-ray powder diffraction pattern comprising peaks at 8.6°, 11.0°, 16.7°, 18.7°, 19.3°, 20.6°, 21.6°, 22.2°, 24.2°, and 24.4°±0.2° in 2θ.

34. The mesylate salt of claim 31, 32 or 33, wherein said Form I is characterized by an X-ray powder diffraction pattern comprising peaks at 8.6°, 11.0°, 11.9°, 16.7°, 18.7°, 19.3°, 20.6°, 21.6°, 22.2°, 24.2°, and 24.4°±0.2° in 2θ.

35. The mesylate salt of claim 2, wherein the crystalline mesylate salt is crystalline Form I characterized by an X-ray powder diffraction pattern comprising at least three or at least four peaks selected from 8.5°, 18.7°, 20.6°, 21.5°, and 24.4°±0.2° in 2θ.

36. The mesylate salt of claim 35, wherein the crystalline mesylate salt is crystalline Form I characterized by an X-ray powder diffraction pattern comprising peaks at 8.5°, 18.7°, 20.6°, 21.5°, and 24.4°±0.2° in 2θ.

37. The mesylate salt of claim 35, wherein the crystalline mesylate salt is crystalline Form I characterized by an X-ray powder diffraction pattern comprising peaks at 8.5°, 10.9°, 16.7°, 18.7°, 19.2°, 20.6°, 21.5°, 22.1°, 24.1°, and 24.4°±0.2° in 2θ.

38. The mesylate salt of claim 35, wherein the crystalline mesylate salt is crystalline Form I characterized by an X-ray powder diffraction pattern comprising peaks at 6.8°, 8.5°, 10.9°, 11.7°, 11.9°, 13.5°, 14.6°, 15.2°, 16.7°, 17.6°, 17.9°, 18.7°, 19.2°, 19.5°, 19.6°, 20.3°, 20.6°, 21.0°, 21.5°, 21.7°, 22.0°, 22.1°, 22.7°, 23.7°, 24.1°, 24.4°, 24.9°, 25.4°, 25.7°, 25.8°, 26.2°, 26.8°, 28.0°, 29.4°, 30.0°, 31.0°, 32.7°, 33.1°, 33.5° and 35.7° 0.2° in 2θ.

39. The mesylate salt of any one of claims 31-38, wherein said Form I is characterized by an X-ray powder diffraction pattern substantially similar to FIG. 4A.

40. The mesylate salt of any one of claims 31-39, wherein said Form I is further characterized by a differential scanning calorimeter (DSC) thermogram comprising an endotherm onset at 33.2° C.±2° C., an endotherm onset at 210.4° C.±2° C., and an exotherm onset at 229.1° C.±2° C.

41. The mesylate salt of any one of claims 31-40, wherein said Form I is further characterized by a thermogravimetric analysis (TGA) substantially similar to FIG. 4B.

42. Crystalline Form A of Compound (I) represented by the following structural formula: 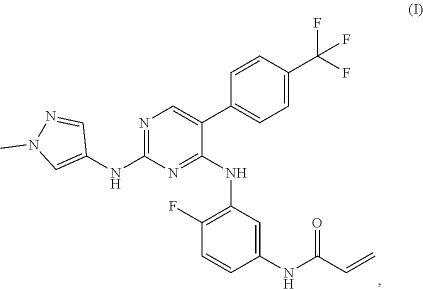 [SMILES: C=CC(=O)Nc1ccc(F)c(Nc2nc(Nc3cnn(C)c3)ncc2-c2ccc(C(F)(F)F)cc2)c1]
wherein said Form A is characterized by an X-ray powder diffraction pattern comprising peaks at 5.7°, 6.0° and 6.2°±0.2 in 2θ.

43. The crystalline Form A of claim 42, wherein said Form A is characterized by an X-ray powder diffraction pattern comprising at least three or at least four peaks selected from 5.7°, 6.0°, 6.2°, 6.5° and 19.6°±0.2° in 2θ.

44. The crystalline Form A of claim 42, wherein said Form A is characterized by an X-ray powder diffraction pattern comprising peaks at 5.7°, 6.0°, 6.2°, 6.5° and 19.6°±0.20 in 2θ.

45. The crystalline Form A of claim 42, wherein said Form A is characterized by an X-ray powder diffraction pattern comprising peaks at 5.7°, 6.0°, 6.2°, 6.5°, 16.9°, 19.6°, 22.4°, 23.7°, 24.9° and 25.2°±0.20 in 2θ.

46. The crystalline Form A of claim 42, wherein said Form A is characterized by an X-ray powder diffraction pattern comprising peaks at 5.7°, 6.0°, 6.2°, 6.5°, 9.7°, 13.0°, 15.8°, 16.9°, 17.1°, 17.3°, 18.8°, 19.5°, 19.6°, 22.4°, 22.7°, 23.7°, 24.9° and 25.2°±0.20 in 2θ.

47. The crystalline Form A of any one of claims 42-46, wherein said Form A is characterized by an X-ray powder diffraction pattern substantially similar to FIG. 5A.

48. The crystalline Form A of any one of claims 42-47, wherein said Form A is further characterized by a differential scanning calorimeter (DSC) thermogram comprising an endotherm onset at 263.3° C.±2° C.

49. The crystalline Form A of any one of claims 42-48, wherein said Form A is further characterized by a thermogravimetric analysis (TGA) substantially similar to FIG. 5B.

50. Crystalline Form B of Compound (I) represented by the following structural formula: 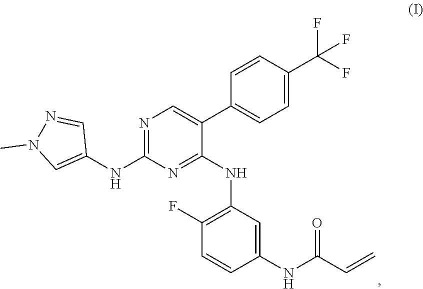 [SMILES: C=CC(=O)Nc1ccc(F)c(Nc2nc(Nc3cnn(C)c3)ncc2-c2ccc(C(F)(F)F)cc2)c1]
wherein said Form B is characterized by an X-ray powder diffraction pattern comprising at least three or at least four peaks at 5.2°, 5.3°, 6.1°, 18.5° and 24.4° 0.20 in 2θ.

51. The crystalline Form B of claim 30, wherein said Form B is characterized by an X-ray powder diffraction pattern comprising peaks at 5.2°, 5.3°, 6.1°, 18.5° and 24.4°±0.2° in 2θ.

52. The crystalline Form B of claim 50 or 51, wherein said Form B is characterized by an X-ray powder diffraction pattern comprising peaks at 5.2°, 5.3°, 6.1°, 15.6°, 18.5°, 18.7°, 19.5°, 22.5°, 24.4° and 26.1°±0.2° in 2θ.

53. The crystalline Form B of claim 50, 51 or 52, wherein said Form B is characterized by an X-ray powder diffraction pattern comprising peaks at 5.2°, 5.3°, 6.1°, 8.1°, 9.1°, 13.8°, 14.2°, 15.6°, 16.6°, 17.3°, 18.5°, 18.7°, 19.5°, 21.9°, 22.5°, 23.1°, 24.4°, 24.9°, 25.4°, 26.1°, 26.2°, 26.6° and 27.1°±0.2° in 2θ.

54. The crystalline Form B of any one of claims 50-53, wherein said Form B is characterized by an X-ray powder diffraction pattern substantially similar to FIG. 6A.

55. The crystalline Form B of any one of claims 50-54, wherein said Form B is further characterized by a differential scanning calorimeter (DSC) thermogram comprising an endotherm onset at 262.2° C. 2° C.

56. A pharmaceutical composition comprising the mesylate salt of any one of claims 1-41 or the crystalline form of any one of claims 42-55, and a pharmaceutically acceptable carrier.

57. A method of treating a disease associated with an epidermal growth factor receptor (EGFR) family kinase in a subject in need thereof, comprising administering to the subject a therapeutically effective amount of the mesylate salt of any one of claims 1-41 or the crystalline form of any one of claims 42-55.

58. The method of claim 57, wherein the disease in the subject is characterized by an EGFR mutation.

59. The method of claim 58, wherein the EGFR mutation comprises a substitution in exon 18, a deletion in exon 19, a substitution in exon 20, an insertion in exon 20, a mutation in the extracellular domain, or a substitution in exon 21.

60. The method of claim 59, wherein the EGFR mutation is selected from del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, A767_dupASV EGFR, 773insAH EGFR, M766_A767insAI EGFR, and any combination thereof.

61. A method of treating cancer in a subject in need thereof, comprising administering to the subject a therapeutically effective amount of the mesylate salt of any one of claims 1-41 or the crystalline form of any one of claims 42-55.

62. The method of claim 61, wherein the cancer is bladder cancer, prostate cancer, breast cancer, cervical cancer, colorectal cancer, endometrial cancer, gastric cancer, glioblastoma, head and neck cancer, lung cancer, urothelial cancer, sinonasal cancer, or non-small cell lung cancer.

63. The method of claim 62, wherein the cancer is non-small cell lung cancer, prostate cancer, head and neck cancer, breast cancer, colorectal cancer, or glioblastoma.

64. The method of any one of claims 61-63, wherein the cancer in the subject characterized by an EGFR mutation.

65. The method of claim 64, wherein the EGFR mutation comprises a substitution in exon 18, a deletion in exon 19, a substitution in exon 20, an insertion in exon 20, a mutation in the extracellular domain, or a substitution in exon 21.

66. The method of claim 65, wherein the EGFR mutation is selected from del19/T790M EGFR, L858R/T790M EGFR, L858R EGFR, L861Q EGFR, S768I EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, A767_dupASV EGFR, 773insAH EGFR, M766_A767insAI EGFR, and any combination thereof.
